(19) United States
(12) Patent Application Publication
(10) Pub. No.: US 2016/0299147 A1
(43) Pub. Date: Oct. 13, 2016

(54) SCREENING, DIAGNOSIS, PROGNOSTICATION AND TREATMENT OF OVARIAN CANCER

(71) Applicant: AFG TECHNOLOGIES S.À.R.L., Luxembourg (LU)

(72) Inventor: Saroj Velamakanni, Littleport Ely (GB)

(21) Appl. No.: 14/900,920

(22) PCT Filed: Jun. 26, 2013
(86) PCT No.: PCT/EP2013/063444

§ 371 Date: Dec. 22, 2015

(51) Int. Cl. G01N 33/574; C12Q 1/68; A61K 31/7088

(52) U.S. Cl. CPC G01N 33/57449; A61K 31/7088; C12Q 1/6886; G01N 2333/96491; C12Q 2600/154; C12Q 2600/158

(57) ABSTRACT

Methods and uses relating to diagnosing ovarian cancer or determining the risk of atypical proliferative epithelial lesions or tumours progressing to invasive ovarian cancer or the risk of recurrent non-invasive disease by detecting a loss-of-function-related genetic alteration in the PAPPA gene or the absence or reduced level of functional PAPPA or an increased proportion of mitotic cells (prophase or prometaphase). Therapeutic aspects enable the sensitisation of mitotically delayed ovarian cancer cells to antiproliferative agents, preferably anti-mitotic agents, by restoring normal progression through mitosis, wherein a first therapeutic agent is applied to release ovarian cancer cells from the mitotic block and a second therapeutic agent or therapy affecting proliferating cells is administered to kill the cycling cancer cells.

### FIELD OF THE INVENTION

This invention relates to the use of specific biological markers for screening, diagnosing, prognosticating and treating ovarian cancer. The invention is also directed to the use of these biological markers for the prognostic assessment of proliferative lesions in ovarian tissue, in order to determine the risk of proliferative lesions progressing to invasive ovarian cancer.

### BACKGROUND OF THE INVENTION

Neoplasms and cancer are abnormal growths of cells. Cancer cells rapidly reproduce despite restriction of space, nutrients shared by other cells, or signals sent from the body to stop reproduction. Cancer cells are often shaped differently from healthy cells, do not function properly, and can spread into many areas of the body. Abnormal growths of tissue, called tumours, are clusters of cells that are capable of growing and dividing uncontrollably. Tumours can be benign (noncancerous) or malignant (cancerous). Benign tumours tend to grow slowly and do not spread. Malignant tumours can grow rapidly, invade and destroy nearby normal tissues, and spread throughout the body. Malignant cancers can be both locally invasive and metastatic.

Ovarian cancer is a common form of gynecological cancer arising from the ovary or fallopian tube. More than 90% of ovarian cancers are classified as “epithelial ovarian cancer”, arising from the surface (epithelium) of the ovary. The risk of developing ovarian cancer appears to be affected by several factors, including age, genetics (including mutations in BRCA 1 and BRCA 2), conditions such as infertility and endometriosis and use of post-menopausal estrogen replacement therapy.

Ovarian cancers readily metastasize by shedding cells into the naturally-occurring fluid within the peritoneal cavity. These cells can then implant on other abdominal structures including the uterus, urinary bladder, bowel and the lining of the bowel wall, forming new tumour growths and obstructions before cancer is even suspected.

Diagnosis of ovarian cancer usually involves physical examination, blood tests and trans-vaginal ultrasound. Diagnosis is confirmed with surgery to obtain biopsies. Ovarian cancer can be classified according to the International Federation of Obstetricians and Gynecologists (FIGO) Staging System. Early stage ovarian cancer (I/II) is difficult to diagnose because the symptoms can be subtle and non-specific, therefore it is often not diagnosed until it spreads and advances to later stages (III/IV).

Treatment for ovarian cancer can vary depending on the stage of progression of the cancer. Treatment usually involves surgery, chemotherapy with anti-proliferative drugs, such as taxanes or cisplatin, and sometimes radiotherapy, although radiation therapy is not effective for advanced stages. However, the efficacy of chemotherapy can be reduced due to resistance or desensitization to chemotherapeutic drugs.

Ovarian cancer usually has a poor prognosis. The mortality rates are high because of a lack of any clear early detection or screening test, meaning that most cases are not diagnosed until they have reached advanced stages. More than 60% of women presenting with ovarian cancer have stage III or stage IV cancer, which has already spread beyond the ovaries. The five-year survival rate for all stages of ovarian cancer is 47%. However, for cases where a diagnosis is made early in the disease, when the cancer is still confined to the primary site, the five-year survival rate is 92.7%. Therefore, there is a need for improvements in the detection, prognostication and treatment of ovarian cancer.

Pregnancy Associated Plasma Protein A (PAPPA) was identified in 1974 as one of four proteins of placental origin circulating at high concentrations in pregnant women, and later found clinical utility as a biomarker for Down's syndrome pregnancies. Its biological function remained an enigma for a quarter of a century until it was identified as a protease that regulates IGF bioavailability through cleavage of the inhibitory insulin-like growth factor binding protein-4 (IGFBP-4). Its role as a IGFBP-4 protease in a diverse range of cell types (e.g. fibroblasts, osteoblasts and vascular smooth muscle cells), together with a highly conserved amino acid sequence in vertebrates, indicated that PAPPA serves a basic function beyond placental physiology.

The localisation of the PAPPA gene on chromosome 9 has been shown by Callahan et al. (Oncogene (2003) 22, p.590-601) as a region associated with loss of heterozygosity in ovarian cancer. This study showed that PAPPA loss was present in greater than 70% of the tumour samples tested.

US2005/0272034 (Conover et al) discloses that PAPPA activity is decreased in malignant ovarian cells due to a corresponding increase in expression of the precursor form of eosinophil major basic protein (proMBP), which circulates in complex with PAPPA and inhibits PAPPA carrying out is proteolytic function (cleavage of IGFBP-4). In contrast to this, another publication by the same group (Boldt and Conover; Endocrinology; (2011) 152 (4) pg 1470-1478) describes over-expression of PAPPA in ovarian cancer cells as a promoter of tumour growth in vivo. Therefore, the mechanisms by which PAPPA activity impacts upon tumour growth are not clearly understood in the art.

There is a need for improved methods for the screening, diagnosis, prognostication and treatment of ovarian cancer, and a better understanding of the role of PAPPA as a biomarker of ovarian cancer.

### SUMMARY OF THE INVENTION

The present invention is based on the surprising finding that Pregnancy Associated Plasma Protein A (PAPPA) is required for normal progression through mitosis, and that PAPPA silencing is highly prevalent in invasive ovarian cancer and pre-invasive borderline tumours (termed ‘atypia’) predisposed to becoming invasive. The present invention provides an important understanding to the biological causes of ovarian cancer, and the resistance of ovarian tumours to cell cycle targeted drugs/agents and therapies, and allows screening, diagnosis, prognostication of pre-malignant lesions and treatment of ovarian cancer to be made in a more focussed and effective way.

The understanding that PAPPA is required for normal progression through mitosis, and that the loss of its expression or impaired functioning contributes significantly to the cancerous state, and in particular progression from pre-invasive to invasive cancer, allows diagnosis of ovarian cancer and prognostication of pre-malignant lesions to be made by monitoring PAPPA expression and/or activity levels, and treatment to be given by targeting therapies for increasing endogenous PAPPA levels or mimicking PAPPA function.

According to the first aspect of the invention, a method for screening for ovarian cancer in a subject comprises detecting the presence of a loss-of-function-related genetic alteration in the PAPPA gene or its regulatory or promoter sequences in a sample obtained from the subject, wherein the presence of a genetic alteration is indicative of ovarian cancer.

According to the second aspect of the invention, a method for aiding the diagnosis of ovarian cancer in a patient comprises identifying the proportion of mitotic cells in a sample obtained from the patient that are in prophase or prometaphase and comparing to a pre-determined cut-off value, wherein if the proportion of cells in prophase or prometaphase is the same or greater than the cut-off value, the result is indicative of ovarian cancer.

According to the third aspect of the invention, a method for aiding the diagnosis of ovarian cancer in a patient comprises detecting the presence of a loss-of-function-related genetic alteration in the PAPPA gene or its regulatory or promoter sequences in a sample obtained from the patient, wherein if a genetic alteration is present, the result is indicative of ovarian cancer.

According to the fourth aspect of the invention, a method for determining the risk of (a) an atypical proliferative epithelial lesion or tumour progressing to invasive ovarian cancer and/or (b) the risk of recurrent non-invasive disease in a patient, comprising identifying the proportion of mitotic cells in a sample obtained from the patient that are in prophase or prometaphase and comparing to a pre-determined cut-off value, wherein if the proportion of cells in prophase or prometaphase is the same or greater than the cut-off value there is risk of progression to invasive ovarian cancer and/or the risk of recurrent non-invasive disease.

A fifth aspect of the invention is directed to the use of H3S10ph immuno-detection to aid diagnosis of ovarian cancer in a patient or to determine the risk of an atypical proliferative epithelial lesion or tumour progressing to invasive ovarian cancer and/or risk of recurrent non-invasive disease.

A sixth aspect of the invention is directed to the use of an antibody having affinity for a marker of mitosis in an assay for aiding diagnosis of ovarian cancer or for determining the risk of an atypical proliferative epithelial lesion or tumour progressing to invasive ovarian cancer and/or the risk of recurrent non-invasive disease in a patient.

According to the seventh aspect of the invention, a method for determining the risk of (a) an atypical proliferative epithelial lesion or tumour progressing to invasive ovarian cancer and/or (b) the risk of recurrent non-invasive disease in a patient comprises detecting the presence and/or level of PAPPA in a sample obtained from the patient, wherein if PAPPA is not present, or is present at a reduced level compared to a control, there is the risk of progression to invasive cancer and/or the risk of recurrent non-invasive disease.

According to the eighth aspect of the invention, a method for determining the risk of (a) an atypical proliferative epithelial lesion or tumour progressing to invasive ovarian cancer and/or (b) the risk of recurrent non-invasive disease in a patient, comprising detecting the presence of a loss of function-related genetic alteration in the PAPPA gene or its regulatory or promoter sequences in a sample obtained from the patient, wherein if a genetic alteration is present there is the risk of progression to invasive cancer and/or the risk of recurrent non-invasive disease.

According to the ninth aspect of the invention, a therapeutic regimen for treating or preventing ovarian cancer in a patient comprises (i) administering a first therapy or therapeutic agent which releases mitotically delayed cells; and (ii) administering a second therapy or therapeutic agent, that acts on proliferating cells to disrupt or otherwise inhibit growth of such cells.

A tenth aspect of the invention provides a chemotherapeutic agent that acts on proliferating cells to disrupt or otherwise inhibit the growth of such cells, for use in combination therapy with a drug that releases cells from mitotic delay in the treatment of ovarian cancer.

An eleventh aspect of the invention provides a biological agent that acts on proliferating cells to disrupt or otherwise inhibit the growth of such cells, for use in combination therapy with a drug that releases cells from mitotic delay in the treatment of ovarian cancer.

A twelfth aspect of the invention provides a nanoparticle that acts on proliferating cells to disrupt or otherwise inhibit the growth of such cells, for use in combination therapy with a drug that releases cells from mitotic delay in the treatment of ovarian cancer.

A thirteenth aspect of the invention is directed to the use of an IGF-1 receptor agonist to reduce the invasive capacity of atypical proliferative epithelial lesions or tumours in a patient.

### DESCRIPTION OF THE FIGURES

FIG. 1 shows a multi-step progression model of ovarian tumourigenesis;

FIG. 2 shows mitotic phase distribution in human cancers. (A) shows representative images identifying distinct mitotic phases by phosphohistone H3 (H3S10ph) immunostaining in tissue sections of surgical biopsy specimens (1000× original magnification; scale bar 10 μm). (B) Pie charts showing the percentage of mitotic cells assigned to each mitotic phase in borderline ovarian cancer (n=21 patients), invasive ovarian cancer (n=154 patients), lung cancer (n=43 patients), bladder cancer (n=40 patients), colon cancer (n=59 patients) and lymphoma (n=48 patients). (C) shows photomicrographs of a representative case of ovarian cancer (400× magnification; scale bar 50 μm) which shows a high frequency of early mitotic figures (indicated by arrows) compared to lymphoma and bladder cancer (1000× magnification; scale bar 20 μm) as examples of the group of other cancers showing normal mitotic progression;

FIG. 3 is a receiver operating characteristic (ROC) curve for the prophase/prometaphase fraction, applying a minimum mitotic cell count of n=5. Data shown are area under the curve (AUC). * indicates p<0.0001 compared to a null diagnostics test;

FIG. 4 shows the distribution of the prophase/prometaphase fraction in borderline ovarian cancer, invasive ovarian cancer and other cancers (pooled). * indicates p<0.0001 compared to other cancers (Mann-Whitney test);

FIG. 5 shows enrichment of early mitotic figures in ovarian cancer. Box plot shows the percentage of mitotic cells in prophase/prometaphase in a range of human cancers. Invasive ovarian carcinoma is characterised by a higher proportion of mitotic cells in prophase/prometaphase compared to other tumour types. Data are presented as the median (solid black line), interquartile range (boxed) and range (enclosed by dashed lines). Outlying cases are depicted as isolated points. * indicates p<0.0001 compared to all other invasive samples (Pearson's test with continuity correction);

FIG. 6 shows that acquisition of the mitotic delay phenotype occurs early in multi-step ovarian tumour progression. (A) Bar chart showing the percentage of cases of other cancers (pooled; n=282), borderline ovarian carcinomas (n=21) and invasive ovarian carcinomas (n=154) exhibiting mitotic delay (p<0.0001; Pearson's chi-square test). Cases are defined as delayed if the proportion of mitotic cells in prophase/prometaphase is at least one third. This cut-point was chosen to allow the proportion of specimens in the combined group of other malignancies properly classified as non-delayed (96%) to be approximately equal to the proportion of ovarian cancer specimens properly classified as delayed (87%). Normal ovarian tissue samples did not contain the minimum number of mitotic cells required to make a statistically significant call for mitotic delay (n=5) and were therefore not included in this analysis. (B) Bar chart showing the percentage of mitotic cells in prophase/prometaphase in other cancers, borderline ovarian carcinomas and invasive ovarian carcinomas. (C) shows photomicrographs of representative cases of non-invasive borderline ovarian carcinoma immunostained for H3S10ph showing normal mitotic phase distribution (left panel) and a high proportion of mitotic cells in prophase/prometaphase (right panel) (400× magnification; scale bar 50 μm);

FIG. 7 shows that PAPPA is epigenetically silenced by promoter methylation in ovarian cancer. (A) shows a bar chart of the percentage of normal ovarian tissue (n=15); benign ovarian lesions (n=24), borderline ovarian lesions (n=30) and invasive ovarian carcinomas (n=174) with PAPPA promoter methylation as determined by MethyLight assay. The proportion of methylated cases was different between the sample types at p=0.0029 (Pearson's chi-square test). (B) shows a bar chart of the percentage of cases expressing PAPPA protein as determined by immunohistochemistry (IHC). Positive PAPPA expression was defined as a score of 2 or more based on intensity and distribution. The proportion of cases expressing PAPPA was different between the sources at p<0.0001 (Pearson's chi-square test). (C) shows photomicrographs of representative cases of borderline and invasive ovarian carcinomas with PAPPA expression in non-methylated cases and a lack of PAPPA expression in methylated cases (630× magnification; scale bar 20 μm);

FIG. 8 shows that PAPPA expression is inversely correlated with increasing tumour stage (p=0.036; logistic regression with Wald approximation). Bar chart of percentage of stage 1 (n=51), stage 2 (n=22) and stage 3 (n=59) ovarian carcinomas expressing PAPPA protein as determined by immunohistochemistry. Positive PAPPA expression was defined as a score of 2 or more based on intensity and distribution;

FIG. 9 shows cell growth characteristics and PAPPA expression in ovarian cancer cell lines. (A) Representative phase-contrast image of Caov-3 cells (200× magnification). The population doubling time for this cell line is 50 hours which was calculated using a Countess™ automated cell counter. (B) Flow cytometry profile of untreated Caov-3 cells following PI staining. Data were analysed using Multicycle AV software and is a summary of results obtained from three independent experiments. (C) Representative phase-contrast image of Ovcar-3 cells (200× magnification). The population doubling time for this cell line is 70 hours which was calculated using a Countess™ automated cell counter. (D) Flow cytometry profile of untreated Ovcar-3 cells following PI staining. Data shown are representative of n=3. (E) MethyLight assays were performed on genomic DNA samples isolated from Caov-3 and Ovcar-3 cells. PMR (percentage methylated reference gene) was obtained by dividing the PAPPA:COL2A1 ratio of a sample by the PAPPA:COL2A1 ratio of CpG methylated HeLa genomic DNA (control) and multiplied by 100. Data presented are the average±standard error of mean (SEM) for n=3. * indicates p<0.05 compared to Caov-3 (Student's unpaired t-test). (F) qRT-PCR was used to measure the relative levels of PAPPA mRNA in Caov-3 and Ovcar-3 cells. PAPPA expression was normalised to the endogenous control GAPDH. Data shown are normalised to relative mRNA expression (RQ) of Caov-3±RQ minimum and RQ maximum and are representative of n=3. * indicates p<0.05 compared to Caov-3 (Student's unpaired t-test). (G) Western blot image of Caov-3 and Ovcar-3 crude cellular fractions probed with rabbit polyclonal anti-PAPPA antibody. β-actin loading controls are shown in the lower panel. (H) The percentage of mitotic cells in prophase/prometaphase in Caov-3 and Ovcar-3 cells assessed by H3S10ph immunostaining. Data presented are the average±SEM for n=3. * indicates p<0.05 compared to Caov-3 (Student's unpaired t-test);

FIG. 10 shows that PAPPA knockdown in Caov-3 cells increases the number of cells accumulating in early mitosis and renders Caov-3 cells more invasive. (A) qRT-PCR analysis of Caov-3 cells transfected with negative control siRNA or PAPPA silencer validated siRNA. Data were normalised to the endogenous control GAPDH for determination of relative mRNA expression (RQ)±RQ minimum and RQ maximum, and are representative of results obtained from two independent experiments. * indicates p<0.05 compared to negative control siRNA (Student's unpaired t-test). (B) Western blot image of Caov-3 cells transfected with negative control siRNA or PAPPA siRNA. The membrane was probed with rabbit polyclonal anti-PAPPA antibody. β-actin loading controls are shown in the lower panel. (C) Representative images (200× magnification) of Caov-3 cells transfected with negative control siRNA or PAPPA siRNA, immunostained with H3S10ph antibody and assigned to distinct mitotic phases as indicated by key. (D) The percentage of mitotic cells in prophase/prometaphase in negative control siRNA or PAPPA siRNA transfected Caov-3 cells. Data presented are the average±SEM, of n=5. * indicates p<0.05 compared to negative control siRNA (Student's unpaired t-test). (E) Number of invading Caov-3 cells as determined from stained membrane insert of a Boyden chamber treated with negative control siRNA compared to PAPPA siRNA transfected cells. Data shown are the average±SEM for n=3. * indicates p<0.05 compared to negative control siRNA (Student's unpaired t-test);

FIG. 11 shows that PAPPA overexpression in Ovcar-3 cells decreases the number of cells accumulating in early mitosis. (A) qRT-PCR analysis of Ovcar-3 cells transfected with negative control (empty plasmid) or PAPPA expressing plasmid. Data were normalised to the endogenous control GAPDH. Data shown are normalised to relative mRNA expression (RQ) of negative control±RQ minimum and RQ maximum and are representative of n=3. * indicates p<0.05 compared to negative control (Student's unpaired t-test). (B) Western blot image of Ovcar-3 cells transfected with negative control or PAPPA expressing plasmid. The membrane was probed with rabbit polyclonal anti-PAPPA antibody. β-actin loading controls are shown in the lower panel. (C) Representative images (200× magnification) of Ovcar-3 cells transfected with negative control or PAPPA expressing plasmid, immunostained with H3S10ph antibody, and assigned to distinct mitotic phases as indicated by key. (D) The percentage of total mitotic cells in prophase/prometaphase in negative control or PAPPA cDNA transfected Ovcar-3 cells. Data shown are the average±SEM for n=4. * indicates p<0.05 compared to negative control (Student's unpaired t-test);

FIG. 12 shows that IGF-1R inhibition in Caov-3 cells increases the proportion of mitotically delayed cells and renders cells more invasive. (A) Representative images (200× magnification) of untreated Caov-3 cells or Caov-3 cells treated with IGFR blocking antibody (100 ng/ml), immunostained with H3S10ph antibody and assigned to distinct mitotic phases as indicated by key. (B) The percentage of mitotic cells in prophase/prometaphase in untreated or Caov-3 cells treated with IGFR blocking antibody (100 ng/ml). Data presented are the average±SEM for n=3. * indicates p<0.05 compared to untreated Caov-3 cells (Student's unpaired t-test) (C) Representative images (100× magnification) of untreated Caov3 cells or Caov-3 cells treated with IGFR blocking antibody from a stained membrane insert from a Boyden chamber assay. (D) Number of invading cells in untreated Caov-3 cells compared to Caov-3 cells treated with IGFR blocking antibody as determined by Boyden chamber assay. Data shown are the average±SEM for n=4. * indicates p<0.05 compared to untreated Caov-3 cells (Student's unpaired t-test);

FIG. 13 shows that ovarian cancer cells expressing PAPPA protein are sensitive to paclitaxel. (A) Phase-contrast images (200× magnification) of Caov-3 cells: untreated cells; ethanol treated (control) cells; or cells treated with IGF-1 (100 ng/ml), paclitaxel (5 nM), or IGF-1+paclitaxel. (B) XTT cell viability assays were performed on Caov-3 cells, which were untreated or treated as described above. Data shown are a summary of results (mean±SEM) obtained from four independent experiments. * indicates p<0.05 compared to untreated (Student's unpaired t-test). (C) The ApoTarget™ Quick Apoptotic DNA Ladder Detection assay was carried out on Caov-3 cells, which were untreated or treated as described above;

FIG. 14 shows that exogenous IGF-1 reverses the mitotic delay phenotype in ovarian cancer cells not expressing PAPPA protein and makes the cells less invasive. (A) Representative images (200× magnification) of untreated Ovcar-3 cells or IGF-1 (100 ng/ml) treated Ovcar-3 cells, immunostained with H3S10ph antibody and assigned to distinct mitotic phases as indicated by key. (B) The percentage of mitotic cells in prophase/prometaphase in untreated Ovcar-3 cells or IGF-1 treated Ovcar-3 cells. Data presented are the average±SEM for n=3. * indicates p=0.055 compared to untreated (Student's unpaired t-test). (C) Representative images (100× magnification) of untreated Ovcar-3 cells or IGF-1 treated Ovcar-3 cells from the stained membrane insert from a Boyden chamber assay. (D) Number of invading cells in untreated Ovcar-3 cells compared to IGF-1 treated Ovcar-3 cells as determined by Boyden chamber assay. Data shown are the average±SEM for n=2; and

FIG. 15 shows that mitotically delayed ovarian cancer cells are sensitive to paclitaxel only after pretreatment with IGF-1. (A) Phase-contrast images (200× magnification) of Ovcar-3 cells: untreated cells; ethanol treated (control) cells; or cells treated with IGF-1 (100 ng/ml), paclitaxel (25 nM), or IGF-1+paclitaxel. (B) XTT cell viability assays were performed on Ovcar-3 cells, which were untreated or treated as described above. Data shown are the average±SEM and representative of n=7. * indicates p<0.05 compared to untreated (Student's unpaired t-test). (C) The ApoTarget™ Quick Apoptotic DNA Ladder Detection assay was carried out on Ovcar-3 cells which were untreated or treated as described above.

### DETAILED DESCRIPTION OF THE INVENTION

US2005/0272034 discloses proMBP as a marker for ovarian neoplasia and describes how increased proMBP expression correlates with decreased PAPPA activity in malignant ovarian cells. Therefore the authors of US2005/0272034 propose reduced proteolytic activity of secreted PAPPA as a marker of ovarian neoplasia. This observation of decreased proteolytic activity of PAPPA in malignant ovarian cells is due to the pro-MBP regulatory loop, i.e. increased expression of pro-MBP results in increased formation of the pro-MBP/PAPPA complex, which prevents PAPPA from carrying out its proteolytic function of cleaving IGFBP-4, hence a corresponding decrease in measured PAPPA activity. Therefore, in the working model described by the authors of US2005/0272034, PAPPA activity is reduced solely as a direct consequence of increased levels of circulating proMBP.

In contrast, the present invention is based on a very different understanding of the role of PAPPA in ovarian cancer. The present inventors have identified that endogenous PAPPA levels and/or functional activity of PAPPA protein is suppressed due to genetic alterations within the PAPPA gene, causing ovarian cells to become temporarily stalled in mitosis. This has implications for the development of ovarian cancer and the progression from non-invasive atypical proliferative epithelial lesions or tumours to ovarian cancer, and also for the desensitisation of cells to anti-proliferative and anti-mitotic agents, such as taxanes, vinca alkaloids or platinum-containing compounds such as cisplatin. This is a significant breakthrough in ovarian cancer detection, prognostication and treatment as it allows “at risk” patients to be identified at an early stage and therapies to be developed in a targeted way.

The following definitions apply to terms used throughout this description and in relation to any of the aspects of the invention described herein.

The terms “patient” and “subject” are used interchangeably herein and refer to any female animal (e.g. mammal), including, but not limited to, humans, non-human primates, canines, felines, rodents and the like, which is to be the recipient of the diagnosis. Preferably, the subject or patient is a human.

The terms “drug” and “agent” are used interchangeably herein, and refer to a chemical or biological substance that exerts an effect on a biological system.

The present invention can utilise naturally-occurring or synthetic nucleic acids. The methods described herein can involve the synthesis of cDNA from mRNA in a test sample or the amplification of naturally-occurring nucleic acids. Portions of cDNA corresponding to genes or gene fragments of interest can be amplified, and the product detected.

As used herein, the term “screening” refers to the process of routinely testing an individual to detect the presence of a disease, in particular ovarian cancer. The subject is preferably an individual who has presented themselves for routine screening for ovarian cancer but who has not experienced or reported any symptoms of ovarian cancer or been motivated to seek medical attention to due symptoms of ovarian cancer (i.e. the subject is asymptomatic for ovarian cancer). Alternatively or additionally, the individual may be at high risk of ovarian cancer, for example due to family history of ovarian cancer.

As used herein, references to “aiding diagnosis” may refer to aiding primary diagnosis, in the context of non-routine testing an individual to detect the presence of a disease, in particular ovarian cancer. In contrast to screening, in the context of diagnosis the patient is an individual who is presenting with symptoms of ovarian cancer or who has been motivated to seek medical attention due to symptoms of ovarian cancer. In certain embodiments, “aiding diagnosis” may also refer to stratification of a patient with a positive diagnosis of ovarian cancer, to further characterise the tumour.

As used herein, “symptoms of ovarian cancer” include, but are not limited to, loss of appetite, indigestion, nausea, excessive gas and a bloated feeling, unexplained weight gain or an increased waist size, swelling in the abdomen associated with shortness of breath, pain in the lower abdomen, changes in bowel or bladder habits, including constipation, diarrhoea or needing to pass urine more often, lower back pain, pain during sex and abnormal vaginal bleeding.

As used herein, the term “a sample” includes biological samples obtained from a patient or subject, which may comprise a tissue sample obtained from the ovary, preferably from the surface epithelium, or fallopian tube, fluid samples including ascites, peritoneal fluid or washings, ovarian cyst fluid and blood/blood components.

Examples of suitable tissue samples include formalin-fixed paraffin-embedded (FFPE) biopsy and/or resection specimens. These comprise protein, DNA, mRNA and/or miRNA, other cellular and extracellular matter and tumour cells (if present in the subject). In the context of the present invention, tissue samples can be used to determine the PAPPA status of the subject (i.e. determine PAPPA expression/activity levels and presence of loss-of-function genetic alterations) and to identify the mitotic delay phenotype by analysing mitotic phase distribution in dividing cells within the tissue. Methods for taking a sample from a patient (biopsy) are conventional and will be apparent to the skilled person.

Ascites, peritoneal fluid or peritoneal washings and ovarian cyst fluid can comprise free DNA, mRNA and/or miRNA and tumour cells (if present in the subject). These samples can be used to investigate the PAPPA status of a subject. The skilled person will be familiar with standard techniques which are suitable for obtaining these samples from a subject.

As used herein, the term “blood sample” includes blood components, including plasma and serum. Circulating free DNA, mRNA, miRNA and, if present, circulating tumour cells, can be extracted from the blood sample and used to determine the PAPPA status of the subject. The skilled person will be familiar with standard phlebotomy techniques which are suitable for obtaining a blood sample from a subject.

The terms “cancer/cancerous” and “neoplasm/neoplastic” refer to or describe the physiological condition in mammals in which a population of cells are characterised by unregulated cell growth.

The terms “cancer cell” and “tumour cell” are grammatical equivalents referring to the total population of cells derived from a tumour or a pre-cancerous lesion. The terms “tumour” and “neoplasm” are used interchangeably herein and refer to any mass of tissue that results from excessive cell growth, proliferation and/or survival, either benign (noncancerous) or malignant (cancerous), including pre-cancerous lesions.

Some of the methods described herein involve establishing whether PAPPA is present in a sample at reduced levels compared to a control. However, it is also envisaged that PAPPA protein may be present at or near to normal levels, but the expressed protein is inactive or active at reduced levels. Accordingly, all aspects of the invention that involve establishing the presence and/or level of PAPPA in a sample also encompass monitoring the activity of PAPPA. PAPPA activity may be reduced by greater than 20%, 30%, 40%, 50%, 60%, 70%, 80%, 90% or greater than 90% compared to a control. Therefore, all references herein to determining whether PAPPA is present (or is present at a reduced level) compared to a control, encompasses the functional activity of PAPPA. Methods for detecting PAPPA protein and PAPPA activity are described in detail below.

The methods of the invention described herein are carried out ex vivo. For the avoidance of doubt, the term “ex vivo” has its usual meaning in the art, referring to methods that are carried out in or on tissue in an artificial environment outside the body of the patient from whom the tissue sample has been obtained.

The term “mitosis” has its usual meaning in the art. Mitosis is the process by which a eukaryotic cell separates the chromosomes in its nucleus into two identical sets, in two separate nuclei. Mitosis and cytokinesis together define the mitotic (M) phase of the cell cycle. The process of mitosis is characterised into stages corresponding to the completion of one set of activities and the start of the next. These stages are prophase, prometaphase, metaphase, anaphase and telophase.

The term “prophase” has its usual meaning in the art. Prophase refers to the stage where the chromatin in the nucleus becomes tightly coiled, condensing into discrete chromosomes.

The term “prometaphase” has its usual meaning in the art. During prometaphase the nuclear membrane disintegrates and microtubules invade the nuclear space.

The term “proliferating cells” refers to cycling cells that are actively dividing and progressing though interphase (G₁, S and G₂ phases) or M phase (mitosis) of the cell cycle.

As used herein, the term “pre-malignant lesion” refers to a morphologically altered tissue in which cancer is more likely to occur than its apparently normal counterpart. As used herein, “atypical proliferative epithelial lesions or tumours” include pre-malignant borderline tumours and intraepithelial neoplasms. The World Health Organisation (WHO) definition of a borderline tumour is “an ovarian tumour exhibiting an atypical epithelial proliferation greater than seen in the benign counterpart but without destructive stromal invasion”. Accepted synonyms of the term “borderline tumour” include tumour of borderline malignancy, cystadenoma of borderline malignancy, tumour of low malignant potential and atypical proliferative tumour. Examples of atypical proliferative epithelial lesions which are within the scope of the invention include tubal intraepithelial carcinoma, ovarian intraepithelial neoplasia and cortical inclusion cysts. Examples of atypical proliferative epithelial tumours, which are also within the scope of the invention, include usual serous borderline neoplasm and micropapillary variant of serous borderline neoplasm.

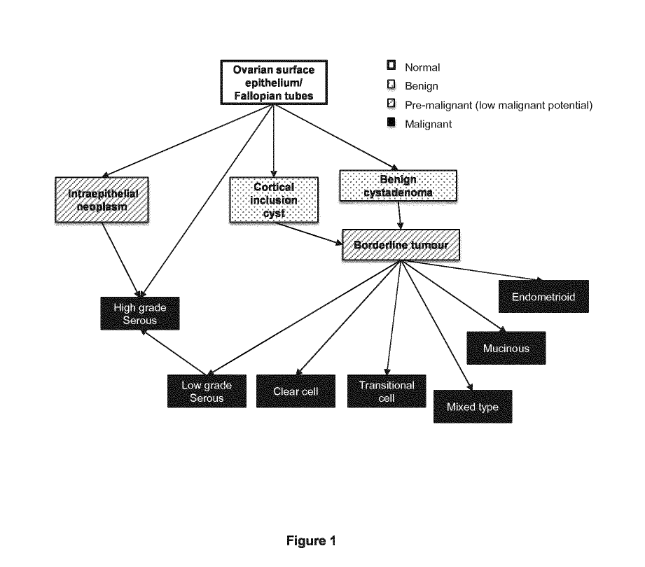

The term “invasive cancer” refers to metastatic cancer, i.e. a primary ovarian lesion or tumour that has spread beyond the epithelium in which it developed, for example into the peritoneal cavity and has invaded and/or is growing in surrounding tissues such as uterus, urinary bladder, bowel and the lining of the bowel wall, forming new tumour growths and obstructions. Other routes of invasion of ovarian cancer include transcoelomic spread, which refers to the spread of a malignancy into body cavities via seeding the surface of the peritoneal, pleural, pericardial, or subarachnoid spaces. FIG. 1 shows the multistep model of ovarian tumourigenesis and distinguishes between normal, benign, borderline and malignant tumours. Invasive cancer is also referred to as infiltrating cancer or malignant cancer. These terms are intended to include all primary invasive surface epithelial ovarian tumours, including adenocarcinomas (such as endometrioid tumour, serous cystadenocarcoinoma, papilliary, mucinous cystadenocarcinoma, clear-cell ovarian tumour, mucinous adenocarcinoma, papilliary serious cystadenocarcinoma), squamous cell carcinomas (epidermoid), germ cell tumours (such as teratoma and dysgerminoma) and other carcinomas such as sex cord-stromal tumours.

As used herein, the phrase “risk of invasive ovarian cancer” refers to the risk of progression from atypical proliferative epithelial lesions/tumours to invasive cancer. Accordingly, the present invention can characterise a patient as at higher or lower risk of developing invasive cancer or recurrence of non-invasive cancer, depending on the mitotic phenotype of the cells. If the delayed mitotic phenotype is identified, the patient is at higher risk, whereas if the delayed mitotic phenotype is not identified, the patient is at lower risk.

As used herein, the phrase “risk of recurrent non-invasive disease” refers to the risk of in situ non-invasive atypical proliferative epithelial lesions/tumours recurring within the ovarian tissue of the patient. Non-invasive proliferative lesions/tumours may recur at the same location or at a different location within the ovarian tissue of the patient. The risk of recurrent non-invasive disease includes the risk of relapse, i.e. the risk of a subject developing non-invasive atypical proliferative ovarian lesions/tumours after convalescence or apparent recovery.

The terms “immunoassay”, “immuno-detection” and immunological assay” are used interchangeably herein and refer to antibody-based techniques for identifying the presence of or levels of a protein in a sample.

The term “antibody” refers to an immunoglobulin which specifically recognises an epitope on a target as determined by the binding characteristics of the immunoglobulin variable domains of the heavy and light chains (V_{H}S and V_{L}S), more specifically the complementarity-determining regions (CDRs). Many potential antibody forms are known in the art, which may include, but are not limited to, a plurality of intact monoclonal antibodies or polyclonal mixtures comprising intact monoclonal antibodies, antibody fragments (for example F_{ab}, F_{ab}′, and Fᵣ fragments, linear antibodies single chain antibodies and multi-specific antibodies comprising antibody fragments), single chain variable fragments (scFᵥS), multi-specific antibodies, chimeric antibodies, humanised antibodies and fusion proteins comprising the domains necessary for the recognition of a given epitope on a target. Preferably, references to antibodies in the context of the present invention refer to monoclonal antibodies. Antibodies may also be conjugated to various reporter moieties for a diagnostic effect, including but not limited to radionuclides, fluorophores or dyes.

The term “epitope” refers to the portion of a target which is specifically recognised by a given antibody. In instances where the antigen is a protein, the epitope may be formed from either a contiguous or non-contiguous number of amino acids (‘linear’ or ‘conformation’ epitopes respectively), whereby in the case of the latter, residues comprising the epitope are brought together in the three-dimensional fold of the polypeptide. An epitope typically comprises, but is not limited to, 3-10 amino acids in specific positions and orientations with respect to one another. Techniques known in the art for determining the epitope recognised by an antibody (specifically whether or not an epitope comprises a given residue) include but are not limited to, site-directed mutagenesis or the use of suitable homologous proteins to the target protein, in combination with techniques for determining specific recognition or lack thereof, as exemplified below. By way of example and not limitation, an epitope may be determined as comprising a given residue by comparative analysis with a control comprising specific recognition of the native (non-substituted) target protein by said antibody; wherein diminished binding and/or lack of specific recognition by said antibody when compared with said control identifies a given residue as forming part of an epitope. Furthermore, structural analyses of antibody-target protein complexes via x-ray crystallography and/or nuclear magnetic resonance (NMR) spectroscopy, or suitable derivatives thereof, may also be used to determine the residues which constitute an epitope.

The term “specifically recognises”, in the context of antibody-epitope interactions, refers to an interaction wherein the antibody and epitope associate more frequently or rapidly, or with greater duration or affinity, or with any combination of the above, than when either antibody or epitope is substituted for an alternative substance, for example an unrelated protein. Generally, but not necessarily, reference to binding means specific recognition. Techniques known in the art for determining the specific recognition of a target by a monoclonal antibody or lack thereof include but are not limited to, FACS analysis, immunocytochemical staining, immunohistochemistry, western blotting/dot blotting, ELISA, affinity chromatography. By way of example and not limitation, specific recognition, or lack thereof, may be determined by comparative analysis with a control comprising the use of an antibody which is known in the art to specifically recognise said target and/or a control comprising the absence of, or minimal, specific recognition of said target (for example wherein the control comprises the use of a non-specific antibody). Said comparative analysis may be either qualitative or quantitative. It is understood, however, that an antibody or binding moiety which demonstrates exclusive specific recognition of a given target is said to have higher specificity for said target when compared with an antibody which, for example, specifically recognises both the target and a homologous protein.

As used herein, the term “biological agent” has its usual meaning in the art and refers to any therapeutic entity derived from a biological or biotechnological source or process, including modified derivatives thereof, or one chemically synthesised to be equivalent to a product from said source, process or derivative thereof. Suitable biological agents include a peptide, a protein, an oligonucleotide, a polysaccharide, a cell-based product, a plant or animal extract, a recombinant protein, an antibody of any suitable type, including a monoclonal, a polyclonal antibody, a humanised antibody, a chimeric antibody, and an antibody fragment, an anti-cancer vaccine, blood or blood components (including erythrocytes, leukocytes, plasma and serum), a pro-drug, or combinations thereof.

As used herein, the term “nanoparticle” refers to a particle having at least one dimension of nanometer (10⁻⁹ meters) scale. Generally, but not necessarily, the term refers to a polymer sphere or spheroid having one dimension of less than or equal to about 5000 nm, including, 5, 10, 15, 20, 30, 50, 100, 200, 250, 300, 350, 400, 500, 750 and 1000 nm. The term includes nanoparticles comprising a number of layers and/or regions of different polymers and/or adsorbed agents. Nanoparticles are used in cancer therapy as drug delivery systems. Typically, the nanoparticles are liposomal and/or polymer-drug conjugates, able to target selectively proliferating cancer cells. Nanoparticles can be linked to biological molecules which can act as address tags, to direct the nanoparticles to specific sites within the body and within the nucleus. Common address tags are monoclonal antibodies, aptamers, streptavidin or peptides. Nanoparticles useful in the therapeutic regimen of the invention may carry anti-proliferative agents (e.g. doxorubicin, Doxil™).

As used herein, the term “radiation therapy” (also known as radiation oncology or radiotherapy) has its usual meaning in the art and refers to the medical use of ionizing radiation as part of a cancer treatment regimen to kill malignant cells that are progressing through the cell cycle (i.e. in any phase of the cell cycle). The radiation therapy may be internal or external radiotherapy. External radiotherapy involves targeting doses (or “fractions”) of high-energy beams of radiation, either X-rays or gamma rays, to the tumour. Internal radiotherapy involves positioning the source of radioactivity inside the body close to the tumour. This can be achieved in two ways: by brachytherapy or by radioisotope therapy. Brachytherapy involves placing a solid source of radiation next to a tumour to give a high dose of radiotherapy. Brachytherapy is often used to treat gynaecological cancers by placing the radiation source inside the vagina during the treatment. Radioisotope therapy involves administration of a radioactive substance, a radioisotope, either as an intravenous injection, or as an oral capsule or liquid.

The term “sequentially” is understood to mean that the first and second therapies must exert their respective biological effects in that specific order. The effect of administering the first drug is that the mitotic block is released, thereby enabling mitosis to progress beyond prophase/prometaphase and opening a window of opportunity for the second drug/therapy to target the cell cycle following prophase/prometaphase. The first and second therapies may be administered together, provided that the first therapy takes effect prior to the second. For example, if given together, the second drug may be in a delayed release form, such that it is active only after the mitotic block has been released.

The present invention has identified that suppression of endogenous Pregnancy Associated Plasma Protein A (PAPPA) levels is implicated in the development of malignant ovarian cancer. More particularly, the present invention has identified that suppression of PAPPA is implicated in the local invasiveness of ovarian cancer. This is a significant breakthrough in ovarian cancer detection and treatment as it allows “at risk” patients to be identified and therapies to be developed in a targeted way.

The inventors have now shown that in ovarian tissue PAPPA is required for normal progression through mitosis. The endogenous suppression of PAPPA levels and/or functional activity of PAPPA cause an early mitotic delay phenotype, with cycling cells temporarily stalling in prophase/prometaphase. This can be reversed, for example by increasing the expression and/or activity of the endogenous PAPPA gene, or by introducing artificial constructs which express PAPPA or by means of mimicking PAPPA (e.g. by increasing the local bioavailability of IGF-1 receptor agonists).

Mitotic delay due to PAPPA suppression in ovarian cancer cells appears at first glance to be disadvantageous to tumour growth. However a major biological advantage is conferred to the mitotically delayed, neoplastic ovarian cell through the associated increase in acquiring invasive capacity. In ovarian cancer specimens, mitotic delay due to PAPPA silencing can be detected in virtually all cases of invasive cancer and also in a proportion of non-invasive borderline lesions. The gain in invasive capacity as a consequence of PAPPA loss therefore occurs early in multi-step ovarian tumour progression during the transition from non-invasive to invasive cancer.

A first aspect of the present invention provides a method for screening for ovarian cancer in a subject, comprising detecting the presence of a loss-of-function-related genetic alteration in the PAPPA gene or its regulatory or promoter sequences in a sample obtained from the subject, wherein the presence of a genetic alteration is indicative of ovarian cancer.

Preferably, the loss-of-function-related genetic alteration in the PAPPA gene or its regulatory or promoter sequences is one or more of a point mutation, deletion, insertion, translocation, chromosomal breakage, loss of heterozygosity (LOH) or methylation. Each of these genetic alterations is described in detail below. In order for a single genetic alteration or combination of two or more genetic alterations to be indicative of ovarian cancer in a subject, the alteration must result in loss of functional PAPPA protein.

The sample obtained from the subject is preferably a blood sample, smear sample or peritoneal fluids or washings. Preferably, peritoneal fluids or washings are only used as the sample when screening for ovarian cancer in high risk subjects, such as those with a family history of ovarian cancer or carriers of the BRCA 1 or BRCA 2 genetic mutations.

The presence of PAPPA may be identified using a PAPPA-specific antibody or a probe for the PAPPA gene, mRNA or a specific PAPPA mutation. Suitable methodologies are described in more detail below.

The present invention also provides an ex vivo method for determining whether ovarian cells are stalled in mitosis, by identifying a delayed mitotic phenotype. Identification of this phenotype comprises identifying the proportion of mitotic cells in a biological sample obtained from a patient that are in prophase or prometaphase and comparing to a pre-determined cut-off value.

The pre-determined cut-off value is at least 30% and preferably at least 33% or more. At least five, preferably at least ten, twenty or fifty of the cells within the sample must be undergoing mitosis. If at least 30% of these at least five mitotic cells are identified as being in prophase or prometaphase then the sample is deemed to have a delayed mitotic phenotype.

For example, if five of the cells within the sample are identified as being in mitosis, at least two of these cells must be in prophase/prometaphase in order for the mitotic delay phenotype to be identified. More preferably the proportion of mitotic cells in prophase/prometaphase in a sample from a patient having the delayed mitotic phenotype is greater than 33%, 35%, 40%, 45%, 50%, 55%, 60%, 65%, 70% or more. Typically, a “control” value for untransformed proliferating cells undergoing mitosis and not displaying the mitotic delay phenotype would be approximately 10-25%, e.g. 24% cells in prophase/prometaphase.

If fewer than five cells in a tissue sample are undergoing mitosis then the analysis of the proportion of mitotic cells that are in prophase/prometaphase will not be sufficiently significant to enable the delayed mitotic phenotype to be identified according to the methods of the invention. Therefore, the methods require there to be at least five mitotic cells in the sample being analysed at the time of analysis.

For methods of the present invention involving determination of the mitotic delay phenotype the biological sample is preferably an ovarian or fallopian tube tissue sample.

Detection of whether the cells in a tissue sample are in prophase or prometaphase can be carried out using techniques conventional in the art. For example, immuno-detection techniques using specific antibodies may be used to characterise the mitotic phase of a cell. Immunohistochemistry (IHC) is an immuno-detection technique and refers to the process of detecting antigens in cells of a tissue section by visualising an antibody-antigen interaction. This can be achieved by tagging an antibody with a reporter moiety, preferably a visual reporter such as a fluorophore (termed “immunofluorescence”) or by conjugating an antibody to an enzyme, such as peroxidase, that can catalyse a colour-producing reaction that can be detected and observed.

H3S10 phosphorylation (H3S10ph) is a mitosis-specific modification essential for the onset of mitosis; the phosphorylation of the serine 10 of Histone H3 is important for chromosome condensation. Antibodies specific for H3S10ph are commercially available (e.g. Millipore and Active Motif) as are kits for carrying out mitotic assays.

Alternative markers of mitosis, such as phospho-specific nuclear phospho-laminin are also available commercially and may be utilised in the methods of the invention. Other methods for assessing and characterising the different phases of mitosis, such as the use of specialist stains (e.g. hematoxylin) and visual morphological analysis, are well known in the art, as will be appreciated by the skilled person.

The analysis can be carried out to provide a “snapshot” of the mitotic phase distribution in dividing cells in a tissue sample. In this way, a mitotic phase distribution analysis is obtained which is then used to characterise the proportion of mitotic cells that are in prophase or prometaphase.

According to a second aspect, the present invention provides a method for aiding the diagnosis of ovarian cancer in a patient, comprising identifying the proportion of mitotic cells in a sample obtained from the patient that are in prophase or prometaphase and comparing to a pre-determined cut-off value, wherein if the proportion of cells in prophase or prometaphase is the same or greater than the cut-off value, the result is indicative of ovarian cancer.

The sample is preferably an ovarian or fallopian tube tissue sample.

The proportion of cells that are in prophase or prometaphase is preferably determined using immuno-detection, and preferably immuno-detection is carried out using an H3S10ph antibody. Alternatively, cells can be stained using specialist dyes and a visual assessment of mitotic phase distribution can be made.

In one embodiment, the method according to the second aspect of the invention further comprises detecting the presence of a loss-of-function-related genetic alteration in the PAPPA gene, or its regulatory or promoter sequences, in a sample obtained from the patient, wherein the presence of a loss-of-function-related genetic alteration is indicative of ovarian cancer. As mentioned above in relation to the first aspect of the invention, the loss-of-function-related genetic alteration in the PAPPA gene or its regulatory or promoter sequences is one or more of a point mutation, deletion, insertion, translocation, chromosomal breakage, loss of heterozygosity (LOH) or methylation. In order for a single genetic alteration or combination of two or more genetic alterations to be indicative of a positive diagnosis of ovarian cancer, the alteration must result in loss of functional PAPPA protein. In this embodiment of the method of the second aspect of the invention the sample may be selected from blood, ascites, peritoneal fluid or washings, and ovarian or fallopian tube tissue. Preferably, the sample is an ovarian or fallopian tube tissue sample.

Additionally or alternatively, the method according to the second aspect of the invention may further comprise detecting the presence and/or level of PAPPA in a sample obtained from the patient, wherein the absence of PAPPA, or the presence of PAPPA at a reduced level compared to a control is indicative of ovarian cancer.

Ovarian cancer is indicated if PAPPA is not present or is present at a level less than 90%, 80%, 70%, 60%, 50%, 40%, 30% or 20% compared to the control. Preferably, if the patient has ovarian cancer PAPPA will be present at an amount between 40%-80% of that of the control. Most preferably, PAPPA will be present at a level between 50%-70%, e.g. approx. 60% compared to that of a control.

In this embodiment of the method of the second aspect of the invention the sample is preferably ovarian or fallopian tube tissue. The control is preferably a corresponding sample obtained from a population of untransformed proliferating ovarian cells, or it may be a reference value.

The presence of PAPPA is preferably identified using a PAPPA-specific antibody.

In addition to being used to aid primary diagnosis of ovarian cancer, the methods of the second aspect of the invention may also be used for stratification of a patient with histologically-confirmed ovarian cancer, by characterising the PAPPA status of the tumour. In this embodiment, the method of the second aspect of the invention is carried out using ovarian or fallopian tube tissue obtained from the patient in order to identify the mitotic delay phenotype. If the delay phenotype is identified, the PAPPA status of the tissue can be characterised by identifying the presence of genetic alterations in the PAPPA gene, and/or detecting the presence and/or level of functional PAPPA in the sample as described above.

According to a third aspect, the invention provides a method for aiding diagnosis of ovarian cancer in a patient, comprising detecting the presence of a loss-of-function-related genetic alteration in the PAPPA gene or its regulatory or promoter sequences in a sample obtained from the patient, wherein the presence of a genetic alteration is indicative of ovarian cancer.

The sample is preferably selected from blood, ascites, cystic fluid, peritoneal fluid or washings and ovarian or fallopian tube tissue.

The loss-of-function-related genetic alteration in the PAPPA gene or its regulatory or promoter sequences is preferably one or more of a point mutation, deletion, insertion, translocation, chromosomal breakage, loss of heterozygosity or methylation. In order for a single genetic alteration or combination of two or more genetic alterations to be indicative of a positive diagnosis of ovarian cancer, the alteration must result in loss of functional PAPPA protein. In this embodiment of the method of the second aspect of the invention the sample may be selected from blood, ascites, peritoneal washings, and ovarian or fallopian tube tissue.

The presence of PAPPA may be identified using a PAPPA-specific antibody or a probe for the PAPPA gene, mRNA or a specific PAPPA mutation.

The methods for aiding screening and diagnosis of ovarian cancer according to the first, second and third aspects of the invention can be complemented by existing screening and diagnostic tools that are known in the art. For example, the screening methods of the invention can be complemented by the identification of known biomarkers of ovarian cancer, such as CA125, in a sample obtained from the subject or patient. As the skilled person will be aware, increased levels of the biomarker CA125 are known to correlate with ovarian cancer. Similarly, the methods of aiding diagnosis of ovarian cancer described herein can be complemented by known diagnostic methods, including histopathological assessment, ultrasound, CT and/or MRI scanning, and identification of known biomarkers of ovarian cancer, such as CA125. The present inventors have found that in atypical proliferative epithelial lesions within ovarian tissue, cells that have no or reduced PAPPA levels or activity and are temporarily stalled in mitosis are pre-disposed to developing an invasive character. Therefore, detection of PAPPA de-regulation and mitotic delay in biological samples offers a significant advance in identifying ovarian cancer patients with non-invasive borderline lesions who are at higher risk of developing invasive disease. Accordingly, the present invention can be used to discriminate patients exhibiting non-invasive lesions into those whose lesions are unlikely to progress to an invasive phenotype (and who may not require additional therapy) and those predisposed to the invasive phenotype (and who may therefore require additional therapy).

Therefore, according to a fourth aspect of the invention, a method for determining the risk of (a) an atypical proliferative epithelial lesion or tumour progressing to invasive ovarian cancer and/or (b) the risk of recurrent non-invasive disease in a patient comprises identifying the proportion of mitotic cells in a sample obtained from the patient that are in prophase/prometaphase and comparing to a pre-determined cut-off value, wherein if the proportion of cells in prophase or prometaphase is the same or greater than the cut-off value there is risk of progression to invasive ovarian cancer and/or the risk of recurrent non-invasive disease.

As described above, the cut-off value is preferably at least 30% of the mitotic cells in the sample and in order for the result to be statistically significant at least five of the cells in the sample must be in mitosis. Therefore, if at least 30% of at least five, preferably at least 10, 20 or 50 mitotic cells are identified as being in prophase or prometaphase then the sample is deemed to have a delayed mitotic phenotype. According to this aspect of the invention, if a delayed mitotic phenotype is identified, this indicates that there is the risk of the atypical proliferative epithelial lesion or tumour progressing to invasive ovarian cancer, and/or the risk of recurrent non-invasive disease.

For example, if five of the cells within the tissue sample are identified as being in mitosis, at least two of these cells must be in prophase/prometaphase in order for the mitotic delay phenotype to be identified and/or for risk of the atypical proliferative lesion progressing to invasive cancer and/or the risk of recurrent non-invasive disease to be determined. More preferably the proportion of mitotic cells in prophase/prometaphase in a sample from a patient having the delayed mitotic phenotype is greater than 33%, 35%, 40%, 45%, 50%, 55%, 60%, 65%, 70% or more. Typically, a “control” value for non-invasive healthy proliferating ovarian tissue cells undergoing mitosis and not displaying the mitotic delay phenotype would be approximately 10-25%, e.g. 24% cells in prophase/prometaphase.

If fewer than five cells in a sample are undergoing mitosis then the analysis of the proportion of mitotic cells that are in prophase/prometaphase will not be sufficiently significant to enable the delayed mitotic phenotype to be identified according to the methods of the invention. Therefore, the methods require there to be at least five mitotic cells in the tissue sample being analysed at the time of analysis.

The proportion of cells that are in prophase or prometaphase is preferably determined using immuno-detection, and preferably immuno-detection is carried out using an H3S10ph antibody. Alternatively, cells can be stained using specialist dyes and a visual assessment of mitotic phase distribution can be made.

The sample is preferably ovarian or fallopian tube tissue, which may include peritoneal implants.

The method according to this aspect of the invention may further comprise detecting the presence of a loss-of-function-related genetic alteration in the PAPPA gene, or its regulatory or promoter sequences, in a sample obtained from the patient, preferably an ovarian or fallopian tube tissue sample, which may include peritoneal implants or pelvic lymph node tissue. If a loss-of-function-related genetic alteration is present, there is the risk of progression to invasive ovarian cancer and/or the risk of recurrent non-invasive disease. The loss-of-function-related genetic alteration in the PAPPA gene or its regulatory or promoter sequences is preferably one or more of a point mutation, deletion, insertion, translocation, chromosomal breakage, loss of heterozygosity or methylation. In order for a single genetic alteration or combination of two or more genetic alterations to the risk of progression to invasive ovarian cancer and/or the risk of recurrent non-invasive disease, the alteration must result in loss of functional PAPPA protein.

Additionally or alternatively, the method may further comprise detecting the presence and/or level of PAPPA in a sample obtained from the patient, wherein if PAPPA is not present, or is present at a reduced level compared to a control, there is the risk of progression to invasive ovarian cancer and/or the risk of recurrent non-invasive disease. Preferably, the presence of PAPPA is identified using a PAPPA-specific antibody.

If PAPPA is not present or is present at a level less than 90%, 80%, 70%, 60%, 50%, 40%, 30% or 20% compared to the control then there is the risk of progression to invasive ovarian cancer and/or the risk of recurrent non-invasive disease. Preferably, in order to identify that there is the risk of progression to invasive ovarian cancer and/or the risk of recurrent non-invasive disease PAPPA will be present at an amount between 40%-80% of that of the control. Most preferably, PAPPA will be present at a level between 50%-70%, e.g. approx. 60% compared to that of a control.

In this embodiment of the fourth aspect of the invention, the sample is preferably selected from ascites, blood, peritoneal fluid or washings, cystic fluid and ovarian or fallopian tube tissue, which may include peritoneal implants and/or pelvic lymph node tissue.

The control is preferably a corresponding sample obtained from a population of untransformed proliferating ovarian cells, or it may be a reference value.

According to a fifth aspect of the invention, immuno-detection is used to diagnose ovarian cancer in a patient or to determine the risk of an atypical proliferative epithelial lesion or tumour progressing to invasive ovarian cancer and/or the risk of recurrent non-invasive disease. Immuno-detection may be carried out using an antibody specific for a marker of any phase of mitosis. Preferred markers of mitosis include H3S10ph and nuclear phospho-laminin. Preferably the immuno-detection is carried out ex vivo.

A preferred embodiment according to this aspect of the invention is directed to use of H3S10ph immuno-detection to aid the diagnosis of ovarian cancer in a patient or to determine the risk of an atypical proliferative epithelial lesion or tumour progressing to invasive ovarian cancer and/or the risk of recurrent non-invasive disease.

According to a sixth aspect of the invention, an antibody having affinity for a marker of mitosis is used in an assay for aiding diagnosis of ovarian cancer or for determining the risk of an atypical proliferative epithelial lesion or tumour progressing to invasive ovarian cancer and/or the risk of recurrent non-invasive disease in a patient. Preferably, the marker of mitosis is selected from H3S10ph and nuclear phospho-laminin.

The ‘assay’ is preferably one of the methods for aiding diagnosis of ovarian cancer or for determining the risk of an atypical proliferative epithelial lesion or tumour progressing to invasive ovarian cancer and/or the risk of recurrent non-invasive disease in a patient described herein.

Preferably, the antibody used according to the fifth and/or sixth aspects of the invention is detectably-labelled, for example by conjugation to radionuclides, fluorophores or dyes. Furthermore, the antibody preferably has an epitope binding affinity in the range of 10⁶-10⁹ M⁻¹, as determined using the methods described herein.

According to a seventh aspect of the invention, a method for determining the risk of (a) an atypical proliferative epithelial lesion or tumour progressing to invasive ovarian cancer and/or (b) the risk of recurrent non-invasive disease in a patient comprises detecting the presence and/or level of PAPPA in a sample obtained from the patient, wherein if PAPPA is not present, or is present at a reduced level compared to a control, there is the risk of progression to invasive cancer and/or the risk of recurrent non-invasive disease.

The presence and/or level of PAPPA in the sample may be determined using the methods described herein. For example, PAPPA may be identified using a PAPPA-specific antibody or a probe for the PAPPA gene, mRNA or a specific PAPPA mutation.

If PAPPA is not present or is present at a level less than 90%, 80%, 70%, 60%, 50%, 40%, 30% or 20% compared to the control then there is the risk of progression to invasive ovarian cancer and/or the risk of recurrent non-invasive disease. Preferably, in order to identify that there is the risk of progression to invasive ovarian cancer and/or the risk of recurrent non-invasive disease PAPPA will be present at an amount between 40%-80% of that of the control. Most preferably, PAPPA will be present at a level between 50%-70%, e.g. approx. 60% compared to that of a control.

The sample used to determine the risk of an atypical proliferative epithelial lesion or tumour progressing to invasive ovarian cancer, i.e. (a), is preferably selected from ascites, blood, cystic fluid, peritoneal washings and ovarian or fallopian tube tissue, which may include peritoneal implants and/or pelvic lymph node tissue. The sample used to determine the risk of recurrent non-invasive disease, i.e. (b), is preferably ovarian or fallopian tube tissue, which may include peritoneal implants and/or pelvic lymph node tissue. Preferably, the tissue exhibits proliferative pre-malignant epithelial tumour or lesions, which preferably comprise borderline tumours and/or intraepithelial neoplasms.

The control is preferably a corresponding sample obtained from a population of untransformed proliferating ovarian cells, or it may be a reference value.

According to the eighth aspect of the invention, a method for determining the risk of (a) an atypical proliferative epithelial lesion or tumour progressing to invasive ovarian cancer and/or (b) the risk of recurrent non-invasive disease in a patient comprises detecting the presence of a loss-of-function-related genetic alteration in the PAPPA gene or its regulatory or promoter sequences in a sample obtained from the patient, wherein if a genetic alteration is present there is the risk of progression to invasive ovarian cancer and/or the risk of recurrent non-invasive disease.

The loss-of-function-related genetic alteration in the PAPPA gene or its regulatory or promoter sequences is preferably one or more of a point mutation, deletion, insertion, translocation, chromosomal breakage, loss of heterozygosity or methylation. In order for a single genetic alteration or combination of two or more genetic alterations to the risk of progression to invasive ovarian cancer and/or the risk of recurrent non-invasive disease, the alteration must result in loss of functional PAPPA protein.

The patient sample used in both (a) and (b) is preferably ovarian or fallopian tube tissue, which may include peritoneal implants and/or pelvic lymph node tissue. In a preferred embodiment, the tissue exhibits proliferative pre-malignant epithelial tumours or lesions, preferably wherein the proliferative pre-malignant epithelial tumours or lesions comprise borderline tumours and/or intraepithelial neoplasms.

The present invention also allows conventional anti-proliferation therapies or therapeutic agents to be given, but increases the effectiveness of these. Many therapies including chemotherapeutic and biological agents, nanoparticles and radiotherapies, act on proliferating cells by targeting a specific phase of the cell cycle, including interphase (G₁, S and G₂ phases) and M phase (mitosis). These therapies and therapeutic agents are less effective when the cancer cell has temporarily stalled in mitosis. By administering agents that allow the proliferating cancer cells to progress through mitosis and divide into two daughter cells which subsequently traverse through the cell cycle, conventional anti-proliferation, and preferably anti-mitotic, therapies/therapeutic agents, can then act on the cells. Therefore, the cancerous ovarian cells cannot avoid the therapy or therapeutic agent due to mitotic inactivity.

Accordingly, a ninth aspect of the invention provides a therapeutic regimen (or method) for treating or preventing ovarian cancer in a patient. The regimen comprises: (i) administering a first therapy or therapeutic agent which releases mitotically delayed cells, and therefore promotes unperturbed transit through prophase, prometaphase, metaphase, anaphase, telophase, cytokinesis and the following interphase; and (ii) administering a second therapy or therapeutic agent, that acts on proliferating cells, and preferably acts on cells in mitosis, to disrupt or otherwise inhibit growth of such cells. The second therapy or therapeutic agent may specifically target cells in G₁, S, G₂ or M phase of the cell cycle. Preferably, the second therapy or therapeutic agent targets cells at a stage of mitosis after prophase/prometaphase.

This aspect of the invention is based upon the observation that mitotically-dividing cells that become stalled in prophase/prometaphase (termed herein “mitotic block”) are not sensitive to therapies that target proliferating cells, and in particular, anti-mitotic therapies that are active against a dividing cell at a stage in mitosis after prophase/prometaphase. Therefore, by first administering to a patient a therapy or therapeutic agent which releases mitotic cells from the mitotic block, the cells are able to progress through normal M phase (i.e. from prophase/prometaphase to metaphase, anaphase, telophase and cytokinesis), thereby becoming sensitive to subsequently/sequentially administered therapies that are active against a proliferating cell, preferably at a stage in mitosis after prophase/prometaphase.

The first therapy or therapeutic agent is preferably a therapeutic agent selected from PAPPA protein, or a nucleic acid encoding functional PAPPA, an IGF-1 receptor agonist, preferably exogenous IGF-1, or a demethylation agent.

IGF-1 (insulin-like growth factor-1) receptor agonists are defined as agents that are capable of binding to and activating the IGF-1 receptor. Examples of IGF-1 receptor agonists suitable for use as the first drug according to the therapeutic regimen of the invention include exogenous IGF-1.

Suitable demethylation agents for use as the first therapy according to the therapeutic regimen of the invention include decitabine (5-aza-2′-deoxycytidine), farazabine, azaytidine (5-azacytidine), histone deacetylase inhibitors (such as hydroxamic acids (e.g. trichostatin A)), cyclic tetrapeptides (e.g. trapoxin B), depsipeptides, benzamides, electrophilic ketones, aliphatic acid compounds (e.g. phenylbutyrate and valproic acid), hydroxamic acids (e.g. vorinostat, belinostat and panobinostat), benzamides and phenylbutyrates.

The second therapy or therapeutic agent may be a compound or molecule that targets proliferating cells, and may be a chemotherapeutic drug, a biological drug or a nanoparticle-based therapeutic agent. Alternatively or additionally, the second therapy may be radiation therapy. Preferably the second therapy or therapeutic agent targets proliferating cells at a stage of mitosis after prophase/prometaphase.

In one embodiment, the second therapy or therapeutic agent is a chemotherapeutic drug, or a combination of chemotherapeutic drugs. The chemotherapeutic drug or drugs is/are preferably selected from one or more of the following classes of chemotherapeutic drugs: taxanes, platinum-containing chemotherapeutic agents, vinca alkaloids, alkylating antineoplastic agents, nucleoside analogue drugs, topoisomerase I/II inhibitors, podophyllotoxins, camptothecins, folate antimetabolites, anthracyclines, tetrahydroisoquinoline alkaloids, PLK1 inhibitors and aurora kinase inhibitors. More specifically, the chemotherapeutic drug is preferably selected from one or more of the drugs paclitaxel (Taxol™, Abraxane™), paclitaxel poliglumex, carboplatin (Paraplatin™), docetaxel (Taxotere™), doxorubicin (Doxil™, Caelyx™, Myocet™), gemcitabine (Gemzar™), cisplatin (Platin™), topotecan (Hycamtin™), oxaliplatin (Eloxatin™), fluorouracil, leucovorin-modulated 5-fluorouracil, podofilox (Condylox™), etoposide (Etopophos™), vinorelbine (Navelbine™), ifosfamide (Ifex™), pemetrexed (Alimta™), trabectedin (Yondelis™), tirapazamine, vintafolide, karenitecan and sapacitabine.

Alternatively, the second therapy or therapeutic agent may be a biological agent. If the second therapeutic agent is a biological agent, it is preferably selected from a peptide, a protein, an oligonucleotide, a polysaccharide, a cell-based product, a plant or animal extract, a recombinant protein, an antibody of any suitable type, including a monoclonal, a polyclonal antibody, a humanised antibody, a chimeric antibody, and an antibody fragment, an anti-cancer vaccine, blood or blood components (including erythrocytes, leukocytes, plasma and serum), a pro-drug, or combinations thereof.

In a preferred embodiment, the biological agent is a polyclonal antibody, preferably conjugated to a chemotherapeutic agent as defined above.

If the biological agent is an antibody, it will have specificity for a proliferating ovarian cancer cell. The antibody may itself be the therapeutic agent, or may be a carrier, bringing a therapeutic agent, such as a chemotherapeutic agent, to the site of action. In another example, the antibody may be conjugated to a radionuclide, to deliver the radionuclide to the site of proliferating ovarian cancer cells where it can exert its beneficial effect.

Examples of suitable biologic drugs for use in the therapeutic regimen of the invention include farletuzumab (MORAb-003), which is a monoclonal antibody that acts against proliferating cells in S phase of the cell cycle by targeting folate receptor-alpha.

Alternatively or additionally, the second therapy or therapeutic agent may comprise a nanoparticle. The nanoparticle may encapsulate chemotherapeutic agents, radionuclides or metal nanoparticles.

According to the therapeutic regimen of the invention, radiation therapy may be administered to the patient in combination with the second therapy, or as the second therapy.

The first and second therapies/therapeutic agents may be administered separately, sequentially or together, provided that the second drug or therapy takes effect after the first has released the mitotic delay. If the treatment regimen involves administering two therapeutic agents (i.e. drugs), they are preferably administered sequentially, with the second agent administered after the first, at a time interval sufficient for the first agent to take effect. If the treatment regimen involves administering radiation therapy, it is preferably administered subsequent to administration of the first agent, at a time interval sufficient for the first drug to take effect. If both a second therapeutic agent and radiation therapy are administered, the second agent may be administered before or after or during a course of radiation therapy.

Prior to administering the first and second therapies/therapeutic agents according to the therapeutic regimen of the invention, it is preferable to have diagnosed the patient as having an atypical proliferative epithelial lesion or tumour, and to determine whether a patient is likely to be responsive to treatment according to the therapeutic regimen of the invention.

In one embodiment, a suitable patient candidate for treatment according to the therapeutic regimen of the invention is identified by determining whether mitotic cells within an ovarian or fallopian tube tissue sample obtained from the patient are delayed in mitosis (i.e. determining whether the patient exhibits the delayed mitotic phenotype). This is preferably achieved by carrying out the method described herein.

Therefore, according to one embodiment, the therapeutic regimen comprises an initial step of identifying the mitotic delay phenotype in the patient by: (i) identifying the proportion of mitotic cells in a tissue sample obtained from the patient that are in prophase or prometaphase; and (ii) comparing to a pre-determined cut-off value. The mitotic delay phenotype is identified if the proportion of cells in prophase or prometaphase is greater than a cut-off value. The cut-off value is preferably at least 30% of mitotic cells within the tissue sample, more preferably at least 33% as described earlier. The tissue sample must contain at least five, preferably ten, twenty or fifty, mitotic cells for the result to be statistically relevant, again as described earlier.

In another embodiment, once the mitotic delay phenotype has been identified in the patient, the therapeutic regimen may optionally comprises an additional initial step of identifying whether the patient is a suitable candidate for treatment according to the therapeutic regimen by detecting PAPPA loss or a loss-of-function-related genetic alteration in the PAPPA gene, or its regulatory or promoter sequences, in an ovarian or fallopian tube tissue sample obtained from the patient. This additional step is preferably achieved by carrying out either or both of the methods described in relation to the seventh or eighth aspects of the invention. If a genetic alteration and/or PAPPA loss is detected, the patient is identified as being a suitable candidate for treatment according to the therapeutic regimen of the invention. Preferably, PAPPA loss or the loss-of-function-related genetic alteration in the PAPPA gene or its regulatory or promoter sequences is due to one or more of a point mutation, deletion, insertion, translocation, chromosomal breakage, loss of heterozygosity or methylation.

In a preferred embodiment the patient is identified as being at risk of invasive ovarian cancer according to the methods of the present invention and is then treated using one or more of the therapeutic applications or regimens described herein.

The present invention also envisages treatment to reduce invasiveness of ovarian cancer. As described above, cancer cells stalled in mitosis due to PAPPA suppression can acquire invasive capacity. By releasing the mitotic block, for example by administering exogenous IGF-1 as described above, the cells have reduced invasive capacity, and therefore reduced malignant potential. This will provide a therapeutic benefit to the patient.

Accordingly, a tenth aspect of the invention provides a chemotherapeutic agent that acts on proliferating cells that are actively dividing and progressing though interphase (G₁, S and G₂ phases) or M phase (mitosis) of the cell cycle to disrupt or otherwise inhibit the growth of such cells, for use in combination therapy with a drug that releases cells from mitotic delay in the treatment of ovarian cancer.

Preferably, the chemotherapeutic agent is as defined above in relation to the ninth aspect of the invention. Preferably the combination drug is selected from PAPPA protein, or a nucleic acid encoding functional PAPPA, an IGF-1 receptor agonist, preferably IGF-1, or a demethylation agent. Preferably the treatment is to be administered to a patient diagnosed with an atypical proliferative epithelial lesion or tumour, or to a patient diagnosed with invasive ovarian cancer.

A related eleventh aspect of the invention provides a biological agent that acts on proliferating cells that are actively dividing and progressing though interphase (G₁, S and G₂ phases) or M phase (mitosis) of the cell cycle to disrupt or otherwise inhibit the growth of such cells, for use in combination therapy with a drug that releases cells from mitotic delay in the treatment of ovarian cancer.

Preferably, the biological agent is as defined above in relation to the ninth aspect of the invention. Preferably the combination drug is selected from PAPPA protein, or a nucleic acid encoding functional PAPPA, an IGF-1 receptor agonist, preferably IGF-1, or a demethylation agent. Preferably the treatment is to be administered to a patient diagnosed with an atypical proliferative epithelial lesion or tumour, or to a patient diagnosed with invasive ovarian cancer.

A related twelfth aspect of the invention provides a nanoparticle that acts on proliferating cells that are actively dividing and progressing though interphase (G₁, S and G₂ phases) or M phase (mitosis) of the cell cycle to disrupt or otherwise inhibit the growth of such cells, for use in combination therapy with a drug that releases cells from mitotic delay in the treatment of ovarian cancer.

Preferably, the nanoparticle agent is as defined above in relation to the ninth aspect of the invention. Preferably the combination drug is selected from PAPPA protein, or a nucleic acid encoding functional PAPPA, an IGF-1 receptor agonist, preferably IGF-1, or a demethylation agent. Preferably the treatment is to be administered to a patient diagnosed with an atypical proliferative epithelial lesion or tumour, or to a patient diagnosed with invasive ovarian cancer.

A thirteenth aspect of the invention is directed to the use of an IGF receptor agonist, preferably exogenous IGF-1, to reduce the invasive capacity of atypical proliferative epithelial ovarian lesions in a patient. Preferably, the patient has been characterised as being at risk of progression to invasive ovarian cancer according to any of the methods described herein.

The present inventors have identified that one cause of PAPPA suppression in ovarian cancer cells (or pre-cancerous cells) is due to epigenetic changes, i.e. methylation of its DNA, primarily the PAPPA promoter region. DNA methylation, caused primarily by covalent addition of methyl groups to cytosine within CpG dinucleotides, occurs primarily in promoter regions of genes due to the large proportion of CpG islands found there. Hypermethylation results in transcriptional silencing.

Detecting the presence or absence of cancer by determining the methylation state of specific genes is known (but not in the context of PAPPA), and conventional methods for doing this may be adapted for use in the present invention. For example, methylation-specific PCR (MSP) has been used to determine the methylation status of specific genes. This technique, referred to also as MethyLight is described in Eads et al, Nucleic Acids Res. 2000; 28(8), and Widschwendler et al, Cancer Res., 2004; 64:3807-3813, the content of each of which is incorporated herein by reference. Alternative methods include Combined Bisulphate Restriction Analyses, Methylation-sensitive Single Nucleotide Primer Extension and the use of CpG island microarrays. Commercially available kits for the study of DNA methylation are available. Accordingly, the present invention makes use of conventional methods for determining the methylation state of the PAPPA gene or its regulatory promoter sequences, for the determination of ovarian cancer or the risk of progressing to invasive ovarian cancer in a patient.

MethyLight is a high-throughput quantitative methylation assay that utilises fluorescence-based real-time PCR (TaqMan®) technology that requires no further manipulations after the PCR step. MethyLight is a highly sensitive assay, capable of detecting methylated alleles in the presence of a 10,000-fold excess of unmethylated alleles. The assay is also highly quantitative and can very accurately determine the relative prevalence of a particular pattern of DNA methylation using very small amounts of template DNA.

According to the present invention, MethyLight can be used to determine the methylation state of the PAPPA gene or regulatory or promoter regions in a sample of genomic DNA obtained from the patient. Determination of the methylation state of the PAPPA gene may comprise the following steps:
i. Genomic DNA is extracted from the ovarian tissue sample and treated with sodium bisulfite to convert unmethylated cytosines to uracil residues (methylated residues are protected);
ii. Primers and probes designed specifically for bisulfite-modified DNA, such as those detailed in Table 2 below, are used to amplify the bisulfite-targeted DNA sample. The primer/probe sets used include a methylated set specific for the PAPPA gene and a set specific for a reference gene (COL2A1);
iii. The data are analysed and Ct values are calculated, for example by using ABI Step One Plus software; and
iv. The percentage of fully methylated PAPPA molecules at the specific locus is calculated by dividing the PAPPA:COL2A1 ratio of a sample by the PAPPA:COL2A1 ratio of a positive control sample (for example, Sssl treated HeLa genomic DNA) and multiplying by 100.

Since MethyLight reactions are specific to bisulfite converted DNA, the generation of false positive results is precluded.

Although DNA methylation (hypermethylation) is one cause of PAPPA suppression, there may be other causes. For example, the PAPPA gene (or its regulatory sequences) may be mutated leading to transcriptional silencing. Point mutations, deletions, loss of heterozygosity and translocations may all cause the PAPPA gene to lose transcriptional activity. Without being bound by theory, it is believed that approximately 40% of PAPPA suppression in ovarian tissue is caused by epigenetic changes including hypermethylation. It is believed that the major cause of PAPPA suppression in ovarian tissue is due to loss-of-function genetic changes including point mutations, insertions, deletions, translocation, chromosomal breakage and loss of heterozygosity.

Point mutations are genetic changes where the mutation of a single DNA base to another base can lead to a non-functional protein. These point mutations can be further classified into nonsense mutations, where the mutant gene brings the protein synthesis to a premature halt; missense mutations, where the altered codon results in the insertion of an incorrect amino acid into the protein; and frame-shift mutations, where the loss or gain of one or more nucleotides causes the codons to be misread, resulting in non-functional proteins. There are a variety of methods available for the detection of point mutations in molecular diagnostics. The choice of the method to be used depends on the specimen being analysed, how reliable the method is, whether the mutations to be detected are known before analysis and the ratio between wild-type and mutant alleles.

Denaturing gradient gel electrophoresis is a suitable technique for mutation detection, particularly for point mutations. A prolonged (48 hr) proteinase K digestion method or DNA easy kit (Qiagen) can be used to extract genomic DNA. Double stranded DNA (PCR fragments of 1 kb) can be generated by multiplex PCR reaction covering the whole of the PAPPA coding region. In order to increase the efficiency of detection GC clamps can be attached to one of the PCR primers. The DNA can then be subjected to increasing concentrations of a denaturing agent like urea or formamide in a gel electrophoresis set up. With increasing concentrations of denaturing agent domains in the DNA will dissociate according to their melting temperature (Tm). DNA hybrids of 1 kb usually contain about 3-4 domains, each of which would melt at a distinct temperature. Dissociation of strands in such domains results in the decrease of electrophoretic mobility, and a 1bp difference is sufficient to change the Tm. Base mismatches in the heteroduplices lead to a significant destabilisation of domains resulting in differences in Tm between homoduplex and heteroduplex molecules. The homo and heteroduplices will be detected by silver staining after gel electrophoresis. This method offers the advantage that 100% of point mutations can be detected when heteroduplices are generated from sense and antisense strands (Cotton R G, Current methods of mutation detection, Mutat Res 1993; 285: 125-44).

Alternative methods available for the detection of point mutations include PCR-single stranded conformation polymorphism, heteroduplex analysis, protein truncation test, RNASE A cleavage method, chemical/enzyme mismatch cleavage, allele specific oligonucleotide hybridisation on DNA chips, allele specific PCR with a blocking reagent (to suppress amplification of wild-type allele) followed by real time PCR, direct sequencing of PCR products, pyrosequencing and next generation sequencing systems.

Other loss-of-function genetic changes that contribute significantly to PAPPA suppression in ovarian tissue are chromosome changes including gene deletions/insertions, wherein a gene or a cluster of genes is lost from, or inserted into in a chromosome, and translocations, which involve breakage and/or exchange of chromosomal fragments.

The technique of pyrosequencing can be used for detection of insertions, deletions, frame-shift mutations. Pyrosequencing is based on the sequencing-by-synthesis principle. In this method a single-stranded PCR/RT-PCR fragment is used as a template for the reaction. During the process of DNA replication after nucleotide incorporation, released PPi (inorganic phosphate) is converted to light by an enzymatic cascade; ATP sulfurylase which converts PPi to ATP in the presence of APS. This ATP would further drive the luciferase-mediated conversion of luciferin to oxyluciferin that generates visible light, which can be detected by a CCD sensor and is visible as a peak in the pyrogram (Ronaghi, M., Uhlen, M., and Nyren, P, A sequencing method based on real-time pyrophosphate, Science; 1998b 281: 363-365). The light signal generated is linearly proportional to the nucleotides incorporated.

A prolonged (48 hr) proteinase K digestion method or DNA easy kit (Qiagen) can be used to extract genomic DNA from the patient tissue sample. PCR and sequencing primers for the PAPPA gene can be designed for use in pyrosequencing. PCR products can be bound to streptavidin-sepharose, purified washed and denatured using NaoH solution and washed again. Then the pyrosequencing primer can be annealed to the single-stranded PCR product and the reaction carried out on, for example, a Pyromark ID system (Qiagen) according to the manufacturer's instructions.

Other methods available for detecting insertions/deletions and frame-shift mutations are big dye terminator sequencing, next generation sequencing systems and heteroduplex analysis using capillary/microchip based electrophoresis.

Another major contributor to PAPPA suppression in ovarian tissue is loss of heterozygosity. All cells contain two copies of somatic genes—one copy inherited from each parent. If a cell develops a mutation in one of the alleles of a tumour suppressor gene like PAPPA, loss of the remaining allele (termed “loss of heterozygosity” or “LOH”) can initiate tumourigenesis.

Loss of heterozygosity (LOH) can be measured using various techniques, including the following:

### Southern Blotting

Tumour and reference control DNA is isolated and digested with restriction enzymes. Digested DNA is subjected to Southern blot. ³²P-labelled DNA probes are designed to bind to restriction fragment length polymorphisms (RFLP). DNA probes are hybridised to the blot and then visualised by autoradiography. The advantage of Southern blotting is the frequency of false positives or negatives is lower than other techniques. The disadvantages are that this technique is low throughput and the amount of DNA required. If DNA is from FFPE tissues the fragmented nature of this DNA may inhibit the ability to be digested by restriction enzymes. Also, newer technologies described below have replaced radioactive labels with fluorescent labels.

### PCR-Based Microsatellite Analysis

Primers are designed to amplify the microsatellite loci. For example, suitable primers for the amplification of KLF4 gene mapping within the FRA9E CFS regions are: 5′ CAGAGGAGCCCAAGCCAAAGAG 3′ (SEQ ID NO. 1) and 5′ CACAGCCGTCCCAGTCACAGT 3′ (SEQ ID NO. 2). One primer from each pair is labelled with T4 kinase and γ [³²P]ATP. PCR amplification is performed using DNA, labelled primer and unlabelled primer. PCR samples are separated by formamide and urea polyacrylamide gel electrophoresis. After fixing and drying, the gel is exposed to X-ray film. Bands are analysed by laser densitometry and the imbalance is defined as the ratio of allele intensities in the tumour sample relative to the ratio of alleles in normal DNA. The advantage of this technique is that it can analyse DNA from fresh tissue, FFPE tissue, and cells. However, DNA from FFPE tissue may be degraded and therefore too short to be amplified by PCR. The disadvantage of this technique is the potential for false positives due to mis-priming. The technique is labour intensive and requires radioactive labels. This technique is usually carried out together with QRT-PCR for e.g for 16 genes in the FRA9E site lost in ovarian cancers. PAPPA is one of the 16 genes found in this site.

### Fluorescence In Situ Hybridisation (FISH)

Samples from FFPE, frozen tissue sections, or cells are fixed and permeabilised. Oligonucleotide pairs are hybridised to target RNA. The use of multiple fluorescent-labelled probes enables multiplexing of targets. Samples are viewed under a fluorescence microscope. Alternatively, to detect DNA a metaphase spread from cells is generated and repetitive DNA sequences are blocked. A probe is designed to hybridise to the target and tagged with fluorophores, targets for antibodies, or with biotin. Samples are viewed under a fluorescence microscope. The advantage of FISH is that it detects balanced translocations. Cells are individually analysed and therefore a heterogeneous population or contaminating normal cells would not affect the analysis. The disadvantages of FISH are that it is labour intensive and in order to target DNA, and metaphase spreads need to be generated from cells.

### Comparative Genomic Hybridisation (CGH)

CGH technology can detect DNA copy number changes across the entire genome by comparing the hybridisation intensity of the subject samples compared to a reference sample. The DNA is fluorescently labelled and mixed with unlabelled human cot-1 DNA to supress repetitive sequences. The DNA mix is hybridised to a normal metaphase spread. The ratio of subject and reference binding is determined by epifluorescence microscopy. The resolution is quite low; to detect a single copy loss the region must be at least 5-10 Mb and amplification changes less than 1 Mb are undetectable. There have been advances to improve the resolution by using array CGH (aCGH) instead of metaphase spreads. The aCGH uses similar technology to microarrays. High resolution aCGH are able to detect structural variations of a resolution of 200 bp. However, aCGH has proved difficult to use with DNA from FFPE. Also, aCGH can require large quantities of subject DNA.

### SNP Microarray

Various SNP microarray technologies are available commercially, e.g. from Affymetrix and Illumina, and can be used in the present invention. The Affymetrix technology uses the same basic principles as DNA microarray: solid surface DNA capture, DNA hybridisation and fluorescence microscopy. The Illumina platform uses oligonucleotide bound BeadArray in micro-wells on either fibre optic bundles or planar silica slides. Normal and tumour DNA is fluorescently labelled and bound to predesigned SNP arrays. For each SNP, four to six probes are used. The fluorescent intensity of DNA bound to perfect match probes against mismatch probes is analysed. SNP microarray platforms can be utilised for high-throughput LOH detection. Microarrays can be used for DNA or RNA-analysis applications. The accuracy of SNP calls are based upon the model used to analyse the data including the different definitions of LOH thresholds. The frequently used Hidden Markov Model-based approaches (Green et al., BMC Cancer; 2010, 10:195) allow detection of LOH from hemizygous deletion of single alleles, but cannot detect copy-number neutral LOH.

### Next Generation Sequencing

Next generation sequencing technology is moving to replace SNP-arrays for the detection of LOH. Whole genome sequencing, targeted resequencing of a gene of interest, or exome sequencing of a gene of interest can be applied to detect SNPs. A range of tumour DNA, or tumour DNA compared to reference DNA can be sequenced by synthesis or by ligation technology. The DNA sequence is read by fluorescence or a change is pH, depending on the instrument used. The advantages of next generation sequencing are that it is high throughput, fast and has a low cost per base. The disadvantages are that the whole gene of interest may not be covered due to repetitive sequences or high GC content. The accuracy of SNP calls are based upon the model used to analyse the data including the different definitions of LOH thresholds.

Alternative methods of the invention require determining the presence (or absence) or the level of PAPPA in a patient's sample. This can be carried out by determining protein levels, or by studying the expression level of the gene coding for the protein. As used herein the term “expression level” refers to the amount of the specified protein (or mRNA coding for the protein) in the ovarian tissue sample. The expression level is then compared to that of a control. The control may be a tissue sample of a person that is known to not have cancer or may be a reference value. It will be apparent to the skilled person that comparing expression levels of a control and the test sample will allow a decision to be made as to whether the expression level in the test sample and control are similar or different and therefore whether the patient has or is at risk of invasive ovarian cancer.

Methods of measuring the level of expression of a protein in a biological sample are well known in the art and any suitable method may be used. Protein or nucleic acid from the sample may be analysed to determine the expression level, and examples of suitable methods include semi-quantitative methods such as in situ hybridisation (ISH) fluorescence and in situ hybridisation (FISH), and variants of these methods for detecting mRNA levels in tissue or cell preparations, Northern blotting, and quantitative PCR reactions. The use of Northern blotting techniques or quantitative PCR to detect gene expression levels is well known in the art. Kits for quantitative PCR-based gene expression analysis are commercially available, for example the Quantitect system manufactured by Qiagen. Simultaneous analysis of expression levels in multiple samples using a hybridisation-based nucleic acid array system is well known in the art and is also within the scope of the invention. Mutation-specific PCR may also be used, as will be appreciated by the skilled person.

Immunohistochemical detection of a mitotic marker (such as H3S10ph) can be carried out by preparing formalin-fixed, paraffin-embedded ovarian tissue sections are prepared and mounted on SuperFrost®Plus charged slides. Following heat-mediated epitope retrieval, endogenous peroxidase activity is quenched and the sections are incubated with a first antibody (suitable H3S10ph antibodies are available, for example, from Millipore) which specifically recognises mitotic markers (such as phosphorylated H3S10) within mitotic cells. The section is then further incubated with a polymer-linked secondary antibody and peroxidase which enables a chromogenic signal to develop following addition with DAB, thereby allowing binding of the first antibody to the mitotic marker to be detected visually. The immunohistochemical procedure described above can be fully automated using commercially available immunostainers.

Analysis of mitotic phase distribution in a patent's sample or cultured cell line can be carried out using at least two consecutive serial sections from each sample and at least two cytospin preparations for each cell line or body fluid (e.g. aspirate) immunolabelled as described above and five to twenty high power fields (400× magnification) image captured and a minimum of 5 mitotic cells for each sample used to determine the mitotic phase distribution. All mitotic cells within the captured fields can be classified based on their chromosomal morphology as prophase/prometaphase, metaphase, anaphase and telophase, according to classical morphological criteria. A population of cells is classified as ‘delayed’ if at least 30% of mitotic cells reside in prophase/prometaphase.

PAPPA levels in an ovarian tissue sample can be determined using conventional immunological detection techniques, using conventional anti-PAPPA antibodies. The antibody having specificity for PAPPA, or a secondary antibody that binds to such an antibody, can be detectably-labelled. Suitable labels include, without limitation, radionuclides (e.g. ¹²⁵I, ¹³¹I, ³⁵S, ³H, ³²P or ¹⁴C), fluorophores (e.g. Fluorescein, FITC or rhodamine), luminescent moieties (e.g. Qdot nanoparticles supplied by Quantum Dot Corporation, Palo Alto Calif.) or enzymes (e.g. alkaline phosphatase or horse radish peroxidase).

Immunological assays for detecting PAPPA can be performed in a variety of assay formats, including sandwich assays e.g. (ELISA), competition assays (competitive RIA), bridge immunoassays, immunohistochemistry (IHC) and immunocytochemistry (ICC). Methods for detecting PAPPA include contacting a patient sample with an antibody that binds to PAPPA and detecting binding. An antibody having specificity for PAPPA can be immobilised on a support material using conventional methods. Binding of PAPPA to the antibody on the support can be detected using surface plasmon resonance (Biacore Int, Sweden). Anti-PAPPA antibodies are available commercially (e.g. HPA001667 from Sigma-Aldrich, MA1-46425 (5H9) from Thermo Scientific, OASA03208 from Aviva Systems Biology and A0230 from Dako). The immuno-detection of PAPPA is also disclosed in U.S. Pat. No. 6,172,198, the content of which incorporated herein by reference.

Immunohistochemical detection of PAPPA within an ovarian or fallopian tube tissue sample can be carried out by preparing formalin-fixed, paraffin-embedded ovarian tissue sections mounted on SuperFrost++ charged slides. Following epitope retrieval by proteolytic digestion, endogenous peroxidase activity is quenched and the sections are incubated with a first anti-PAPPA antibody (available, for example, from DAKO). The section is then further incubated with a polymer-linked secondary antibody and peroxidase which enables a chromogenic signal to develop following addition with DAB, thereby allowing binding of the first antibody to the PAPPA protein to be detected visually. The immunohistochemical procedure described above can be fully automated using commercially available immunostainers. Immunocytochemistry (ICC) can be used to detect PAPPA in cells contained in a fluid sample, such as peritoneal fluid or washings, or ascites.

Preferred testing methodologies for different analytes are summarised in Table 1.

TABLE 1

| Analyte | Raw material | Testing Methodology |
|---|---|---|
| Ascites | Tumour cells | PCR/Methylation-specific PCR |
| Cyst fluid | Free DNA, mRNA or | Next generation (high throughput) |
| Peritoneal | miRNA | sequencing |
| fluid or | | ELISA |
| washings | | ICC/Immunofluorescence |
| Smear | | FISH/CISH |
| | | Mass spectrometry |
| | | Micro-chip-based |
| | | immunomagnetic detection |
| Blood, | Circulating tumour cells | PCR/Methylation specific PCR |
| Plasma, | Circulating free DNA, | Next generation (high throughput) |
| Serum | mRNA or miRNA | sequencing |
| | | ELISA |
| | | ICC/Immunofluorescence |
| | | FISH/CISH |
| | | Mass spectrometry |
| | | Nanoparticle-based |
| | | immunomagnetic detection |
| FFPE | Protein | IHC |
| biopsy, | DNA | FISH/CISH |
| Resection | RNA | PCR/Methylation specific PCR |
| specimen | | Next generation (high throughput) |
| | | sequencing |
| | | Mass spectrometry |

PAPPA protein expression can be classified using conventional methods, for example, a combined score for membrane and cytoplasmic staining intensity and staining distribution can be evaluated using the following scoring system: intensity of positive signal is scored as follows: negative (0), no staining is observed; weakly positive (1+), a faint/barely perceptible membrane/cytoplasmic staining is detected; moderately positive (2+), weak staining is detected; strongly positive (3+), strong membrane/cytoplasmic staining is detected. Distribution of staining is scored as follows: focal positivity of less than 10% of cells (1), positivity between 10-50% of cells (2), positivity over 50% of cells (3). Scores for intensity and distribution are combined to give a minimum score of 2 and a maximum score of 6.

For analysis of a relatively small number of PAPPA proteins, a quantitative immunoassay such as a Western blot or ELISA can be used to detect the amount of protein (and therefore level of expression) in a ovarian tissue sample. Semi-quantitative methods such as IHC and ICC can also be used.

To analyse a larger number of samples simultaneously, a protein array may be used. Protein arrays are well known in the art and function in a similar way to nucleic acid arrays, primarily using known immobilised proteins (probes) to “capture” a protein of interest. A protein array contains a plurality of immobilised probe proteins. The array contains probe proteins with affinity for PAPPA.

Alternatively, 2D Gel Electrophoresis can be used to analyse simultaneously the expression level of PAPPA. This method is well known in the art; a sample containing a large number of proteins are typically separated in a first dimension by isoelectric focusing and in a second dimension by size. Each protein resides at a unique location (a “spot”) on the resulting gel. The amount of protein in each spot, and therefore the level of expression, can be determined using a number of techniques. An example of a suitable technique is silver-staining the gel followed by scanning with a Bio-rad FX scanner and computer aided analysis using MELANIE 3.0 software (GeneBio). Alternatively, Difference Gel Electrophoresis (DIGE) may be used to quantify the expression level (see Von Eggeling et al; Int. J. Mol Med. 2001 October; 8(4):373-7.

As explained above, all references herein to determining the presence/level of PAPPA also encompass determining the functional activity of PAPPA. PAPPA activity can be measured using conventional techniques. For example, PAPPA activity can be determined by examining IGFBP-4 proteolytic activity in a sample. Methods for detecting PAPPA activity are disclosed in US patent publication No. 2005/0272034, the content of which is incorporated herein by reference. Alternatively, loss of PAPPA activity may also be determined by mutation-specific PCR analysis.

In one embodiment, PAPPA activity may be detected by screening for proteolytic cleavage of its substrate IGFBP-4 using immunoblotting. PAPPA secreted into the medium can be detected by incubating the media samples in a buffer, such as 50 mM Tris (pH 7.5) supplemented with IGFBP-4. Samples can then be incubated (for example, at 37° C. for 4 hrs) and the proteolytic products detected by immunoblotting using available commercial antibodies against IGFBP-4 protein.

Alternatively, PAPPA activity can be detected by using an ELISA (Enzyme linked immunosorbent assay), wherein specific antibodies against PAPPA are immobilised in the well of a microtitre plate. After washing away unbound protein the activity of PAPPA can be measured using a synthetic substrate which liberates a coloured product only if the primary specific reaction between PAPPA and its antibody has occurred and the bound PAPPA is active. The colour developed is quantified spectrophotometrically using a microplate reader.

Alternatively, the interaction between PAPPA and its substrate IGFBP-4 can also be assessed using Biacore (Surface plasmon resonance technology) or Fluorescence polarisation assay. These methods offer the advantage of being very sensitive and specific and can easily be adapted to develop a high-throughput assay.

As a control for the above described methods a mutant PAPPA protein (E483Q), which is proteolytically inactive, may be used.

In addition to the diagnostic methods of the invention, the present invention provides molecules able to replace or increase PAPPA activity levels/function in a cell, for use in the treatment of ovarian cancer. For example, PAPPA protein, or nucleic acid able to express PAPPA, or an agonist of the IGF receptor (e.g. exogenous IGF-1) may be used to counteract the effect of PAPPA suppression in a patient. Methods for the delivery of proteins or nucleic acids to sites in an organism are well known and may be used in the present invention.

As methylation of DNA is an epigenetic modification, and can be reversed to allow the cells to express PAPPA and progress through a normal mitosis, demethylating drugs are an attractive therapeutic option for promoting mitotic cell division. Therefore, where suppression is caused by hypermethylation of PAPPA DNA, the therapeutic can be:
i. a molecule that reverses the methylation of the PAPPA gene or regulatory or promoter sequences;
ii. an agent comprising a moiety that binds competitively methyl groups and/or prevents methylation at cytosines (i.e. an inhibitor of DNA methylation);
iii. an antagonist/inhibitor of DNA methyl transferase (DMT); or
iv. antisense oligonucleotides against the region of the PAPPA gene promoter comprising a CpG island.

One or more or all of these agents that relate to and/or affect methylation or demethylation at CpG sites on the PAPPA promoter may be used as a therapeutic according to the present invention. Antagonists or inhibitors can be any molecule capable of antagonising or inhibiting the target bio-activity. Therefore, antagonists or inhibitors can be, for example, small molecules, proteins, polypeptides, peptides, oligonucleotides, lipids, carbohydrates, polymers and the like.

Targeting suitable demethylating drugs to the PAPPA gene or promoter sequences is by convention methods and compounds to do this are within the scope of the present invention. Suitable demethylating agents include decitabine (5-aza-2′-deoxycytidine), farazabine, azaytidine (5-azacytidine), histone deacetylase inhibitors (such as hydroxamic acids (e.g. trichostatin A)), cyclic tetrapeptides (e.g. trapoxin B), depsipeptides, benzamides, electrophilic ketones, aliphatic acid compounds (e.g. phenylbutyrate and valproic acid), hydroxamic acids (e.g. vorinostat, belinostat and panobinostat), benzamides and phenylbutyrates.

The demethylating agent can be delivered by any delivery method, including by systemic administration. Delivery can also be via infusion into the peritoneal cavity of a patient. Delivery to the peritoneal cavity may be accomplished, for example, using a delivery tool such as a catheter or cannula and infusing the demethylating agent in a suitable medium or solution directly into the peritoneal cavity to contact target epithelial cells. The amount of the agent can vary, but will be an amount sufficient to target all atypical cells in the epithelium and peritoneal cavity and an amount sufficient to inhibit or reverse DNA methylation on PAPPA promoters expressed in target cells.

In still another approach, expression of the gene encoding endogenous PAPPA can be up-regulated using suitable expression techniques. Known techniques involve the use of genetic constructs which replace the endogenous gene with an artificial alternative. Alternatively, promoter or control sequences may be inserted upstream of the endogenous gene. This may be carried out using conventional methods.

The present invention also provides ovarian cell lines comprising a methylated PAPPA gene promoter, for use in a screening method to detect compounds that up-regulate PAPPA. The cells may be ovarian cancer cells or non-invasive abnormal ovarian cells. The screening method can involve contacting the cell with a potential therapeutic agent and determining whether the agent up-regulates PAPPA in the cell. The agent may, for example, be a demethylating agent, or may be a nucleic acid construct which expresses PAPPA within the cell.

The present invention also provides an isolated genetic construct, for use in the treatment of ovarian cancer, wherein the construct comprises functional PAPPA-expressing nucleic acid, linked operably to regulatory sequences. Such constructs can be prepared using conventional technologies, as will be appreciated by the skilled person. As the PAPPA gene and promoter sequence are known, it will be readily apparent to the skilled person how to prepare a suitable construct. The PAPPA mRNA sequence is identified in NCBI accession number NM_002581.3, the protein sequence is identified in NCBI accession number NP_002572.2. Homologues in human or other species can be found on the NCBI database and on the MitoCheck database (www.mitocheck.org).

PAPPA or other therapeutically-active agents described herein may be formulated in combination with a suitable pharmaceutical carrier, excipient or diluent. Such formulations comprise a therapeutically effective amount of the protein (or other agent), and a pharmaceutically acceptable carrier, excipient or diluent. Such carriers include but are not limited to, saline, buffered saline, dextrose, water, glycerol, ethanol, and combinations thereof. The formulation should suit the mode of administration, and is well within the skill of the art. The invention further relates to pharmaceutical packs and kits comprising one or more containers filled with one or more of the ingredients of the compositions mentioned herein.

Proteins and other compounds of the present invention may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration is also possible.

The dosage range required depends on the choice of protein (or other active), the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Proteins used in treatment can also be generated endogenously in the subject, in treatment modalities often referred to as “gene therapy” as described above. Thus, for example, cells from a subject may be engineered with a polynucleotide, such as a DNA or RNA, to encode a protein ex vivo, and for example, by the use of a retroviral plasmid vector. The cells are then introduced into the subject.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavouring agents may be provided in the pharmaceutical composition. Examples of preservatives include sodium benzoate, sorbic acid and esters of hydroxybenzoic acid. Antioxidants and suspending agents may be also used.

There may be different composition/formulation requirements dependent on the different delivery systems.

Where appropriate, the pharmaceutical compositions can be injected parenterally, for example intravenously, intramuscularly or subcutaneously. For parenteral administration, the compositions may be used in the form of a sterile aqueous solution which may contain other substances, for example enough salts or monosaccharides to make the solution isotonic with blood.

The content of all publications referred to herein is incorporated by reference.

The invention is described with reference to the accompanying drawings, by the following non-limiting examples.

### EXAMPLE

Methods

Physical Units

Throughout this example section, all references to hours, minutes, seconds, milliseconds and millivolts are abbreviated as hrs, min, s, ms and my, respectively.

Tissue Specimens

Formalin-fixed, paraffin-embedded (FFPE) tissue resection specimens and tissue microarrays (TMA) were obtained from commercial sources (Tissue Solutions, Glasgow, UK, and Insight Biotechnology, Wembley, UK, respectively). Samples included invasive ovarian carcinoma (n=188); borderline ovarian cancer (n=38); benign ovarian cancer (n=25); normal ovarian epithelium (n=30); colon adenocarcinoma (n=59); transitional cell carcinoma of the bladder (n=40); penile squamous cell carcinoma (n=33); gastric adenocarcinoma (n=30); malignant melanoma (n=29); small cell lung cancer (n=43); and non-Hodgkin lymphoma (n=48). Histological specimens had been reviewed by three independent qualified pathologists at diagnosis and assessed for histological subtype and nuclear grade according to World Health Organization (WHO) criteria.

Chemicals

Chemicals were purchased from Sigma Aldrich (Dorset, UK) unless otherwise stated.

Cell Culture

Caov-3 cells (ATCC® HTB-75™) were cultured in DMEM (Life Technologies; Cat. No. 31966-021) supplemented with 10% FBS (Life Technologies; Lot #41Q822OK) and GlutaMAX™ (Life Technologies; Cat. No. 35050-038). Ovcar-3 cells (ATCC® HTB-161™) were cultured in RPMI 1640 (LGC Standards; Cat. No. 30-2001) supplemented with 20% FBS (Life Technologies) and 10 μg/ml bovine insulin (Sigma Aldrich, Cat. No. 10516). Cells were cultured at 37° C. with 5% CO₂ and ≧95% humidity. Cells were harvested following incubation with TrypLE™ Express (Life Technologies; Cat. No. 126005-010). Cell density and viability were determined by trypan blue exclusion using a Countess® Automated Cell Counter (Life Technologies; Mod No. C10281). The population doubling time was calculated by PDT=(t2−t1)/3.32×(log n2−log n1), where t is the sampling time and n is the cell density at the time of sampling. Photomicrographs shown were taken using a Panasonic digital camera fitted to a Leica IL-LED microscope. Where indicated, Caov-3 cells were treated with 100 ng/ml IGFR blocking antibody (Millipore; Lot #30080) for 24 hrs, 100 ng/ml IGF-1 (Life Technologies; Lot #73159414A) for 48 hrs or 5 nM paclitaxel (Taxol® Sigma Aldrich; Lot #091M1781V) for 24 hrs. Ovcar-3 cells, where indicated, were treated with 100 ng/ml IGF-1 for 48 hrs or 25 nM paclitaxel for 24 hrs.

PAPPA Overexpression

Full length PAPPA cDNA (NM-002581.3) cloned into pCMV6-XL5 vector available commercially from Origene was used for this experiment. PAPPA cDNA (20 μg) was transfected into 2×10⁶ Ovcar-3 cells using the Neon® Transfection System (Life Technologies; Model No. MPK5000) according to the manufacturer's recommendations with the following optimised settings: two pulses of 1050 mV, 30 ms. 48 hrs post-transfection PAPPA expression was determined by qRT-PCR, immunoblotting and the phenotype established by mitotic phase distribution analysis.

RNA Interference

Knock-down of PAPPA expression was carried out using PAPPA Silencer® Validated siRNA (Life Technologies; Cat. No. 4390817). Non-targeting siRNA Silencer® Select negative control (Life Technologies; Cat. No. 439-0843) was used as a negative control. 100 nM siRNA was transfected into 2×10⁶ Caov-3 cells using the Neon® Transfection System according to the manufacturer's recommendations with the following optimised settings: two pulses of 1150 mV, 30 ms. Cells were incubated for 48 hrs and silencing of PAPPA was assessed by qRT-PCR, immunoblotting and the phenotype established by mitotic phase distribution analysis.

Cell Cycle Analysis

Cells that reached 60-70% confluency were harvested by treatment with TrypLE™ Express. The culture medium supernatant was pooled with the detached cells and centrifuged for 3 min at 194×g (this step was included to ensure retention of all mitotic cells and avoid loss through mitotic shake off). The cell pellet was washed and resuspended in PBS to give a concentration of 2×10⁶ cells/ml. The resuspended cells were transferred to a Coulter Flow cytometry tube (Beckman Counter) and fixed by drop wise addition of 1.5 ml ice-cold 100% ethanol whilst vortexing. The cells were placed on ice for 30 min to fix. Following ethanol fixation, the cells were pelleted by centrifugation for 5 min at 194×g. The supernatant was carefully removed and the cells washed with PBS (added drop-wise whilst vortexing). The cells were finally resuspended in 300 μl DNA Prep PI solution (Beckman Coulter; Cat. No. 6607055) and incubated for 10 min at room temperature in the dark prior to cell cycle analysis on the Navios Flow Cytometer (Beckman Coulter; Ser. No. AN36127). Data presented were analysed using the Multicycle-AV software (Phoenix Flow Systems V328).

Cytospin Preparation

Cells that had reached 60-70% confluency were harvested by treatment with TrypLE™ Express. The culture medium supernatant was pooled with the detached cells and centrifuged for 3 min at 194×g (this step was included to ensure retention of all mitotic cells and avoid loss through mitotic shake off). The cell pellet was washed and resuspended in PBS to give a concentration of 0.5×10⁶ cells/ml. The resuspended cells were cytospun onto Leica Snow Coat glass slides (Leica; Cat. No. 3808100GE) for 5 min at 60×g using a Cytospin 4 cytocentrifuge (Thermo Scientific; Mod. No. A78300101). Cytospins were fixed in 10% neutral buffered formalin for 10 min at room temperature. Slides were either processed immediately or were stored overnight at 4° C. in 10% neutral buffered formalin.

Immunohistochemistry

Section deparaffinisation, antigen retrieval and immunostaining were performed using the Leica Bond-III Autostainer and Bond Polymer Refine Detection kit (Leica; Cat. No. D59800), according to the manufacturer's instructions. Heat-mediated antigen retrieval at pH 6.0 for 30 min was used for both H3S10ph and PAPPA antigens. Primary antibodies were applied for 40 min at the following dilutions: PAPPA (DAKO; Lot #00061479) at 1/200; H3S10ph (Millipore; Lot #2066052) at 1/2000. Cytospin preparations were immunostained using the same protocol without the deparaffinisation step. Incubation without primary antibody was used as a negative control and sections of tonsil and placenta were used as positive controls for H3S10ph and PAPPA antibodies, respectively.

Mitotic Phase Distribution Analysis

Two consecutive serial sections from each FFPE tissue sample and two cytospin preparations for each cell line were immunostained with H3S10ph. Five to 20 high power fields (400× magnification) were image-captured using a Leica DM2500 microscope, Leica DFC295 digital camera and Leica Application Suite software V4.1. A minimum of five mitotic cells for each sample were used to determine the mitotic phase distribution. All mitotic cells within the captured fields were classified based on their chromosomal morphology as prophase/prometaphase, metaphase, anaphase and telophase according to established criteria. Mitotic cells were classified as prophase/prometaphase if chromosome condensation was evident with or without the nuclear envelope intact (prophase or prometaphase, respectively). Metaphase was defined if the sister chromatids showed alignment at the metaphase plate in the centre of the spindle. Anaphase was defined if the sister chromatids were separated into two distinct sets migrating to opposite poles. Telophase was defined by the complete segregation of the sister chromatids and the reformation of the nuclear envelope around each set of chromosomes. A population of cells was classified as ‘delayed’ if at least a third of mitotic cells resided in prophase/prometaphase.

Laser Capture Microdissection

Laser-capture microdissection was performed on a Leica LMD6500 microdissection microscope V7.4.1, using the Leica Application Suite software V4.2, a 355 nm ultraviolet (UV-A) Nd: YAG solid-state laser, and UVI 5x0.12 Microdissection objective (Leica). Formalin-fixed, paraffin-embedded sections (Tissue Solutions, Glasgow, UK) or tissue microarray cores (Insight Biotechnology, Wembley, UK) were cut at 5 μm thickness using a rotary microtome and mounted on PET-Membrane 1.4 μm slides (Leica; Cat. No. 11505151). Sections were baked in a 37° C. oven for 2 hrs and then deparaffinised in xylene, rehydrated through graded alcohols to water, stained with Mayer's haematoxylin for 10 s, washed in tap water and then air-dried for 20 min. Using the Leica Application Suite software V4.2, regions of interest were selected with the drawing tool, cut out by the laser and collected by gravity into the cap of a system-mounted 0.2 ml PCR tube (Greiner Bio-One) containing 50 μl of DNA extraction buffer [PCR buffer (Amplitaq; Cat. No. Y02028) supplemented with 1.5 mM MgCl₂, 0.5% Tween 20 and 0.32 mg/ml Proteinase K (Ambion; Cat. No. AM2546)]. The samples were vortexed, pulse centrifuged, and incubated at 56° C. in a Veriti Thermocycler for a total of 48 hrs with 0.48 μl of (20 mg/ml stock) Proteinase K added after 24 hrs of incubation. After 48 hrs incubation, samples were heated to 99° C. for 15 min to inactivate Proteinase K, then cooled to 4° C. for 10 min, and stored at −20° C. for future use. DNA concentration was determined using Nanovue Plus Spectrophotometer (GE Healthcare; Mod. No. 28956058).

MethyLight Assay

Genomic DNA was extracted from cells using the QIAamp DNA kit (Qiagen; Cat. No. 51304) according to the manufacturer's protocol. DNA isolated was quantified using NanoVue Plus spectrophotometer. For each reaction 400-500 ng of genomic DNA from cell lines or from tissue (using laser capture microdissection) was bisulfite-modified using the EZ DNA Methylation-Gold Kit (Zymo Research; Cat. No. D5006) according to the manufacturer's instructions. Bisulfite-modified DNA was stored at −80° C. until required. CpG methylated HeLa genomic DNA (New England Biolabs; Cat. No. N4007S) was used as positive control. Two sets of primers and probes were designed for bisulfite-modified DNA: a methylated set for PAPPA and collagen 2A1 to normalise for input DNA. Real time PCR reactions were carried out using the TaqMan® Universal PCR Master Mix [No AmpErase® UNG (Life technologies; Cat. No. 00058004345-01)] with 20 ng DNA, 0.3 μM probe and 0.9 μM of both forward and reverse primer. The reactions were carried out on a StepOne Plus Real Time PCR system (Life Technologies; Mod. No. 272006346). The cycling conditions were: 95° C. (10 min), followed by 50 cycles of 95° C. (15 s), 60° C. (1 min). The results of the PCR reaction were analysed (DDC_{T} and RQ calculations) using the StepOne software V2.2. The percentage of methylated PAPPA was calculated by dividing the PAPPA: COL2A1 ratio of the sample by the PAPPA: COL2A1 ratio of the CpG methylated HeLa genomic DNA and multiplying by 100. The abbreviation PMR (percentage methylated reference gene) was used to indicate this measurement. Primers used for this study are shown in Table 2.

TABLE 2

| | Forward primer | Reverse primer | |
|---|---|---|---|
| Gene | sequence 5′-3′ | sequence 5′-3′ | Probe sequence 5′-3′ |
| PAPPA | GCGTGTTTGTGCGAG | CGCCTTCCGAATATACC | 6-FAM-TCGCC |
| | AGTTGT | CATT | CGAATATCTCTACGCCGCT- |
| | (SEQ ID NO. 3) | (SEQ ID NO. 4) | BHQ-1 |
| | | | (SEQ ID NO. 5) |
| COL2A1 | TCTAACAATTATAAA | GGGAAGATGGGATAGA | 6-FAM-CCTT |
| | CTCCAACCACCAA | AGGGAATAT | CATTCTAACCCAATACCTATC |
| | (SEQ ID NO. 6) | (SEQ ID NO. 7) | CCACCTCTAAA-BHQ-1 |
| | | | (SEQ ID NO. 8) |

qRT-PCR Analysis

Total cellular RNA was isolated using the Ambion PureLink RNA Mini kit (Life Technologies; Cat. No. 12183018A), according to the manufacturer's instructions. qRT-PCR reactions were carried out using the TaqMan® RNA-to-C_{T}™ 1-Step kit (Life Technologies; Cat. No. 4392938) according to the manufacturer's instructions. PCR reactions were carried out in StepOne Plus Real Time PCR system (Life Technologies). 400 ng template RNA, final primer concentration of 900 nM and final probe concentration of 250 nM was used in each individual PCR reaction. The probes used in this study were: PAPPA (assay ID Hs01032307_m1 Life Technologies) which spans exons 21-22 and GAPDH (assay ID Hs03929097_g1 Life Technologies) which locates to exon boundary 9 of transcript variant 1 and exon boundary 8 of transcript variant 2. The cycling conditions were: 48° C. (15 min), 95° C. (10 min) followed by 40 cycles of 95° C. (15 s), 60° C. (1 min). The results of the qRT-PCR reaction were analysed (DDC_{T} and RQ calculations) using the StepOne software V2.2.

Immunoblotting

Cells were lysed in crude cellular extraction buffer [10 mM HEPES pH 7.8, 10 mM KCl, 1.5 mM MgCl₂, 0.34 M glucose, 10% Triton X-100 and 1× Complete Protease Inhibitor cocktail (Roche; Cat. No. 04693132001)]. After incubation on ice for 10 min the sample was centrifuged for 5 min at 1,301×g at 4° C. The supernatant containing the crude cellular extract was retained for further analysis. The protein concentration was determined using the Bradford protein assay kit (Pierce; Cat. No. 1856209) according to the manufacturer's protocol. 20-30 μg of crude cellular fractions and MagicMark™ (Life Technologies; Cat. No. LC5602) were separated using Novex° 4-20% Tris-Glycine SDS PAGE (Life Technologies; Cat. No. EC6028). Proteins were transferred from the polyacrylamide gel onto PVDF membrane using the iBlot® dry electroblotting system (Life Technologies; Mod. No. iBlot®). The membrane was blocked for 60 min in PBS supplemented with 10% milk. The membrane was further probed with rabbit polyclonal anti-PAPPA antibody. This step was carried out overnight at 4° C. with gentle agitation. After incubation with the primary antibody, the membrane was washed five times for 10 min with PBS. HRP conjugated secondary goat anti-rabbit antibody (Dako; Cat. No. P0448) in PBS with 10% milk was added to the membrane and incubated for 60min at room temperature. Equal volumes of reagent A and B from ECLSelect™ kit (GE Healthcare; Cat. No. RPN2235) were added to the membrane and incubated for 1 min at room temperature. Images were captured using the GeneGnome chemiluminescent detection system (Syngene; Mod. No. 75000). The membrane was reprobed with anti-β actin antibody (Sigma Aldrich; Lot #121M4846) to ensure equal loading of total protein in each lane.

XTT Viability Assay

Caov-3 or Ovcar-3 cells were plated at a density of 1×10⁴ cells per well into a 96-well tissue culture plate. The cell number was determined using the Countess™ automated cell counter (Life Technologies). Where indicated paclitaxel and IGF-1 were added. The final volume of the tissue culture medium was 0.1 ml. Prior to the experiment for each 96 well plate, 5 ml XTT solution and 0.1 ml activation reagent (New England Biolabs; Cat. No. 9095S) were mixed. 50 μl of the activated solution was added to each well and incubated at 37° C. for 4 hrs. The plates were shaken gently to evenly distribute the dye in the wells. After the 4 hrs incubation the absorbance of the samples was measured using a Spectramax M5e plate reader (Molecular Devices; Mod. No. M5e) at a wavelength of 450 nm.

Analysis of PAPPA Protein Expression

Each FFPE tissue specimen was immunostained for PAPPA and digitally scanned at 200× magnification using the Leica SCN400 scanner. The entire section was viewed using the Leica SlidePath Gateway LAN software V2.0 and given a combined score for intensity and distribution of PAPPA staining. Intensity of positive signal was scored from 0-3+ (0 being negative for PAPPA and 3+ showing the highest staining intensity). Distribution of PAPPA staining was scored from 0-3 (0 being negative for PAPPA, 1 for focal positivity of less than 10% of cells, 2 if between 10-50% of cells were positive and 3 if over 50% of cells showed PAPPA positive staining). Positive PAPPA expression was defined as a combined score of 2 or above.

Invasion Assay

FBS (10%) mediated cell migration through an extracellular matrix was measured by Boyden chamber assay [(BD Biocoat™ Matrigel invasion chamber) BD Biosciences; Cat. No. 354480)] following the manufacturer's instructions. Briefly, Caov-3 cells were transfected with PAPPA siRNA or control siRNA (negative control) as described above. Caov-3 cells were treated with IGFR blocking antibody or untreated as described previously. Ovcar-3 cells were treated with IGF-1 or untreated as described previously. Prior to transfections or treatments the cells were serum starved for 24 hrs. Caov-3 cells were collected in DMEM medium containing 5% BSA, counted and 0.5×10⁵ cells were added to each invasion assay chamber. Ovcar-3 cells were collected in RPMI medium containing 5% BSA, counted and 2×10⁵ cells were added to each invasion assay chamber. After incubation for 48 hrs the invasion chamber inserts were washed with PBS, fixed in 4% formaldehyde for 5 min, stained with 0.01% crystal violet or haematoxylin and cells from random areas on the filters were counted.

Apoptosis DNA Ladder Kit

Caov-3 or Ovcar-3 cells were plated into T75cm² culture flasks in a volume of 25 ml. The cells were seeded at 1×10⁶ cells per flask as determined using the Countess™ automated cell counter (Life Technologies). Where indicated paclitaxel, IGF-1, or IGF-1+paclitaxel were added. Cells were harvested following incubation with TrypLE™ Express (Life Technologies), washed once with PBS and the cell pellet stored at −80° C. The assay was performed using the ApoTarget™ Quick Apoptotic DNA Ladder Detection Kit (Life Technologies; Cat. No. KHO1021) according to the manufacturer's instructions. Briefly, cells were resuspended in 35 μl TE lysis buffer and 5 μl Enzyme A solution. This was mixed with gentle vortexing and incubated at 37° C. for 10 min. 5 μl Enzyme B solution was added and the sample incubated at 50° C. for 30 min. 5 μl Ammonium Acetate solution and 100 μl ice-cold ethanol was added, vortex mixed and stored at −80° C. for 30 min. The sample was centrifuged for 10 min at 17,000×g and the supernatant was removed. The pellet was washed with 0.5 ml 70% cold ethanol and re-centrifuged for 10 min at 17,000×g. The supernatant was removed and the pellet air dried for 10 min at room temperature. DNA was resuspended in 30 μl nuclease-free water (Ambion; Cat. No. AM9937) and was quantified using a NanoVue Plus Spectrophotometer (GE Healthcare). 1 μg of each sample was separated by 1% agarose gel containing 0.2 μg/ml ethidium bromide. Electrophoresed samples were visualised by UV transillumination (UVP BioDoc-It® Imaging System; Mod. No. 95-0452-02).

Statistical Analysis

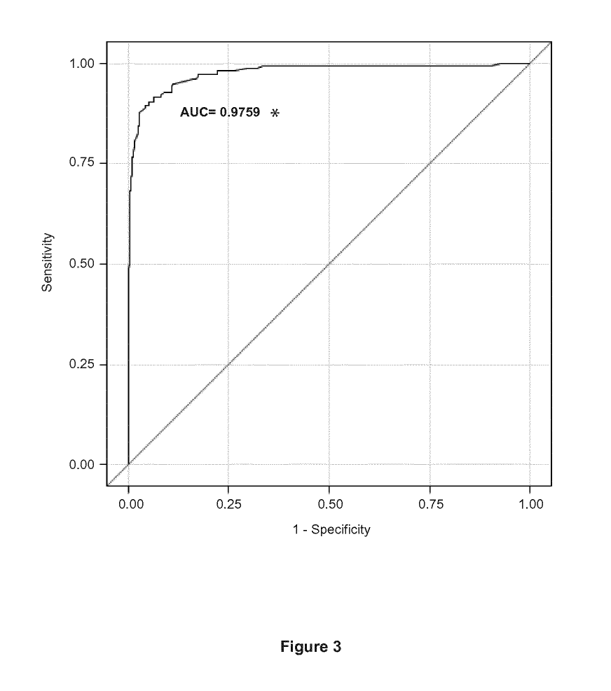

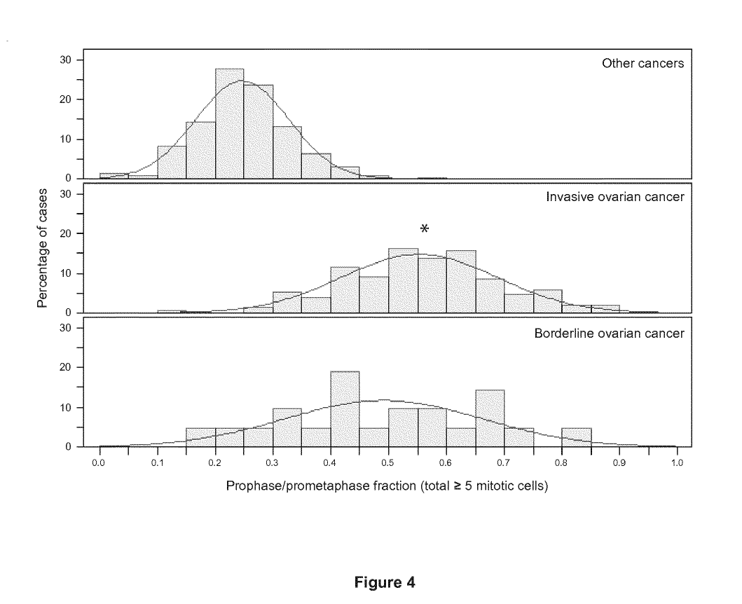

The proportion of mitotic cells in prophase/prometaphase was calculated for each specimen of premalignant and malignant tissue. Receiver Operating Characteristic (ROC) curves for differentiating ovarian cancer from other malignancies (pooled) using the proportion of cells in prophase/prometaphase were constructed for various minimum numbers of mitotic cells. It was clear that the ROC curve was not compromised by letting the minimum requirement be as low as five mitotic cells and was significantly better than a null diagnostic test (p<0.0001) (FIG. 3). The p value was obtained by calculating the area under the curve (AUC) using the trapezoidal method and the result interpreted as a Mann-Whitney statistic with Wald approximation. In the interest of using as many specimens as possible, the evaluability threshold for analysis of mitotic delay was set to at least five mitotic cells observed per specimen. A specimen was declared ‘delayed’ if at least one third of its mitotic cells were in prophase/prometaphase. This requirement was derived by balancing the sensitivity and specificity associated with distinguishing ovarian cancer from other malignancies: 96% of evaluable ovarian cancer specimens had at least one third of their mitotic cells in prophase/prometaphase, while 87% of evaluable other malignancies had less than one third of their mitotic cells in prophase/prometaphase (p<0.0001) (FIG. 4). The proportion of evaluable specimens with mitotic delay was compared between sources of specimen (ovarian cancer, other malignancies) using Pearson's chi-squared test with Yates's continuity correction. The median proportion of mitotic cells in prophase/prometaphase was compared between sources of specimen using the Mann-Whitney test. All significance probabilities reported are two-sided. The association of mitotic delay with tumour differentiation and tumour stage was assessed using a logistic regression test with stage/grade as the continuous variable and Wald approximation. In vitro cell line data were statistically analysed using the Student's unpaired t-test. Results that were statistically significant at p<0.05 are indicated by an asterisk symbol.

Results

Ovarian Cancer is Specifically Enriched in Early Mitotic Figures

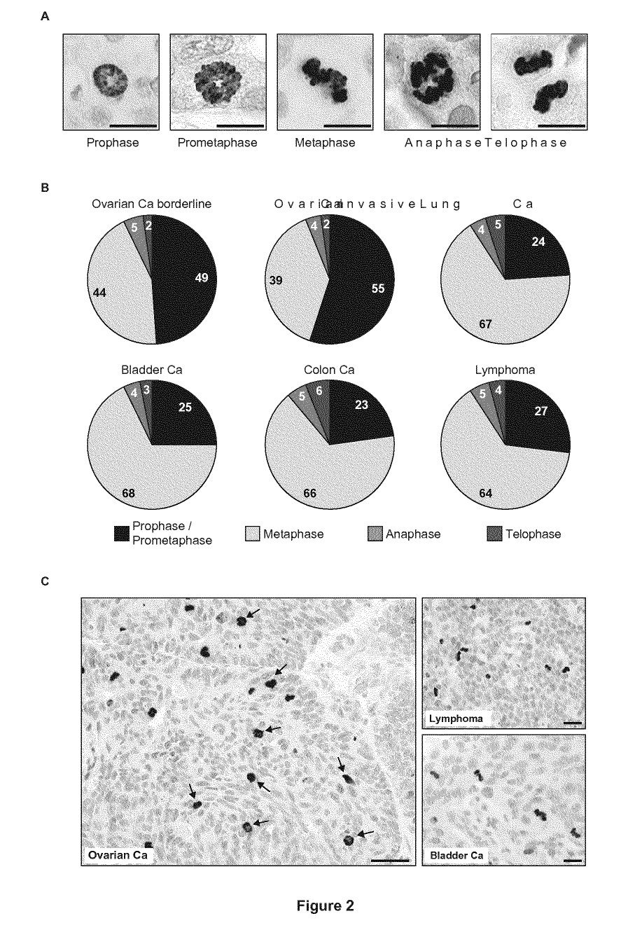

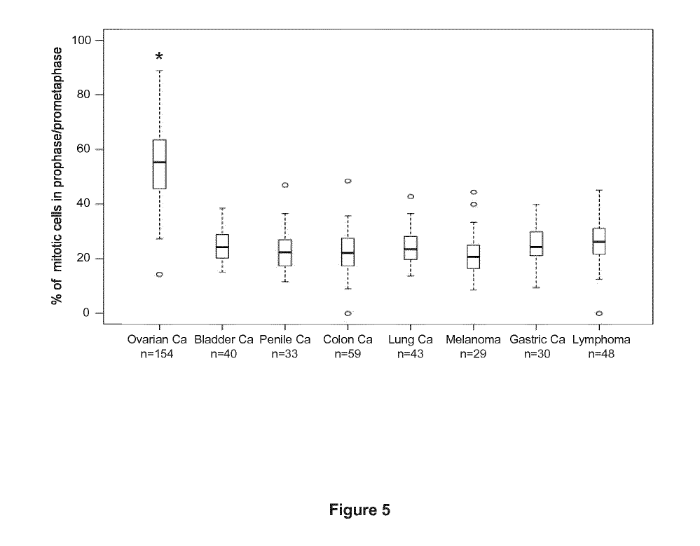

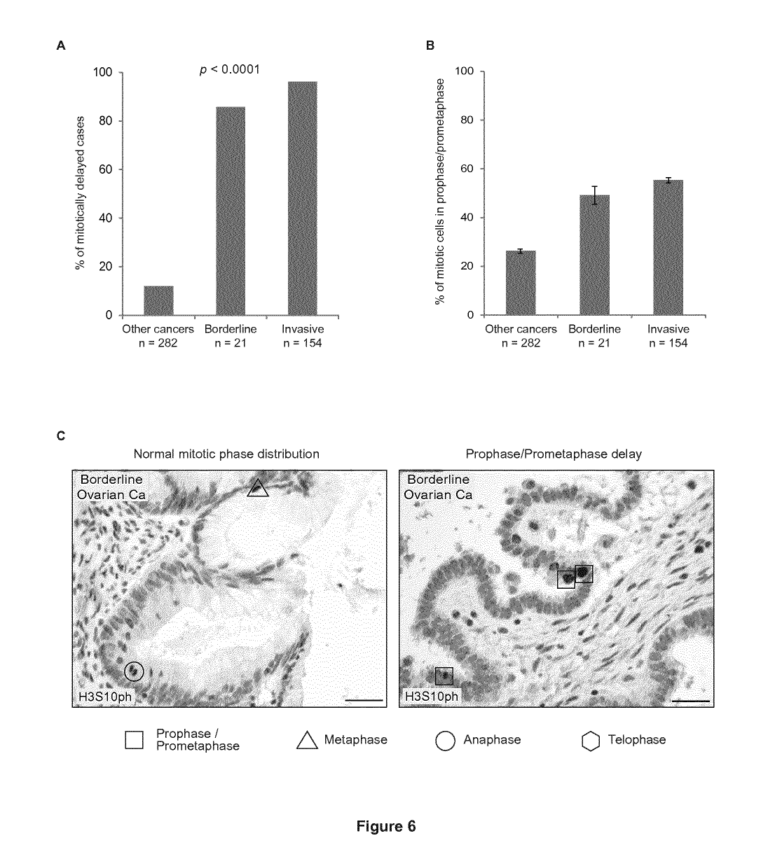

In evaluable tissue specimens from seven common human tumour types, including lung, colon, bladder, penile, melanoma, gastric and lymphatic cancer (n=282 patients), the majority of mitotic cells were in metaphase (FIG. 2 and FIG. 5). In marked contrast, the inventors found a strong prophase/prometaphase enrichment in 96% (148 out of 154 patients) of ovarian cancers compared with only 13% for the combined group of other malignancies (FIG. 5 and FIG. 6A). Cases were defined as displaying an early mitotic delay if at least one third of mitotic cells was in prophase/prometaphase. A minimum count of 5 mitotic cells was shown to be specific by ROC curve analysis (AUC: 0.9759, FIG. 3). The cut-point of one third was chosen to allow the proportion of specimens in the combined group of other malignancies properly classified as non-delayed (87%) to be approximately equal to the proportion of ovarian cancer specimens properly classified as delayed (96%) (see FIG. 4). The mean proportion of mitotic ovarian cancer cells in prophase/prometaphase was 55% (median 55%) compared with 24% (median 24%) in the other malignancies (FIG. 2B-C, FIG. 5 and FIG. 6B), indicating that early mitotic delay is a hallmark of ovarian cancer. Importantly, the inventors found that the mitotic delay phenotype could be detected already in 86% (18 out of 21 patients) of non-invasive borderline lesions, in which the mean proportion of mitotic cells in prophase/prometaphase was 49% (median 48%) (FIG. 2B and FIG. 6C). Thus, the inventors identified an unexpectedly high frequency of early mitotic figures (prophase/prometaphase) in nearly all tested ovarian cancers, revealing a formerly unrecognised delay in mitotic progression in this tumour type.

PAPPA Loss is Linked to Mitotic Delay in Ovarian Cancer

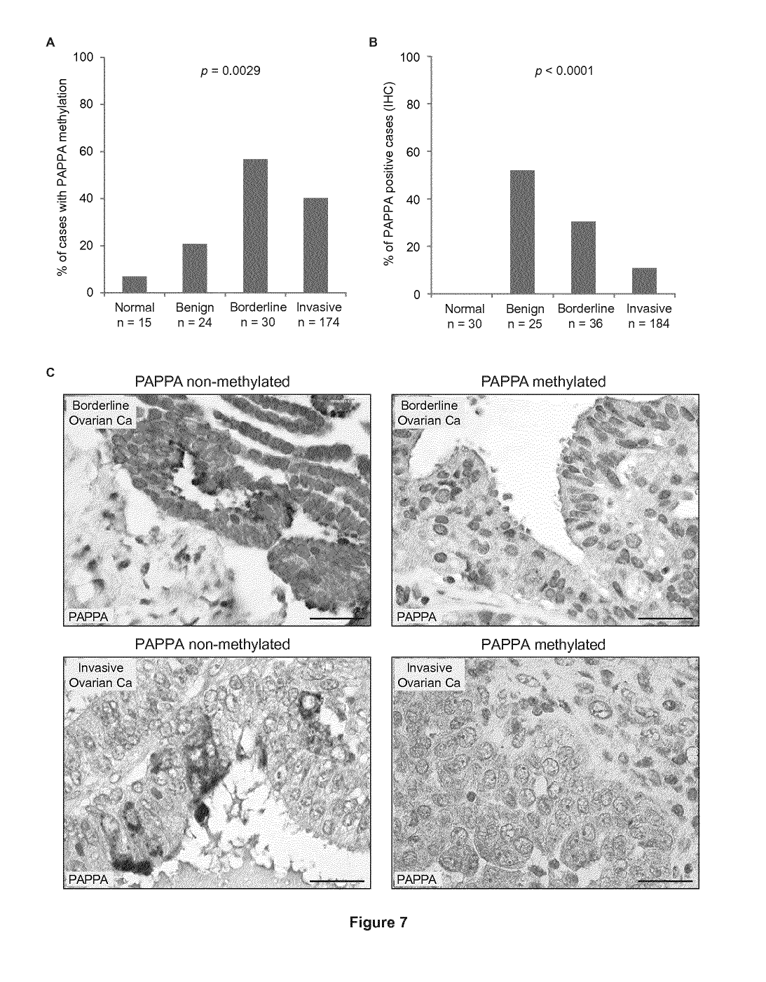

Since promoter methylation represents a common mechanism for loss of tumour suppressor genes during cancer development, the inventors hypothesised that epigenetic silencing of PAPPA could be linked to the mitotic delay phenotype, which proved to be the case in ovarian cancer. MethyLight assays (Widschwendter, M. et al. Cancer research (2004) 64, 3807-3813) showed that PAPPA is strongly hypermethylated in the 5′ regulatory region of the gene in invasive ovarian cancer and in non-invasive borderline ovarian cancer (FIG. 7A). Forty % (70 out of 174 patients) of invasive ovarian cancers and 57% (17 out of 30 patients) of borderline ovarian cancers showed PAPPA hypermethylation (defined as PMR>1; percentage methylated reference gene). In contrast, PAPPA was non-methylated in 93% (14 out of 15 patients) of normal ovarian samples and 79% (19 out of 24 patients) of benign ovarian lesions (FIG. 7A; note that 14 cases of invasive ovarian cancer, 8 cases of borderline ovarian cancer, 1 benign ovarian case and 15 cases of normal ovary were not available for MethyLight analysis due to poor preservation of DNA). This made PAPPA a strong candidate to explain the prophase/prometaphase delay found in ovarian cancer. To test if PAPPA promoter methylation indeed caused gene silencing, the inventors used a commercially available anti-PAPPA antibody (DAKO) for immunostaining of FFPE tissue sections. Immunostaining profiles showed that indeed 89% (58 out of 65 patients) of borderline and invasive ovarian cancers with methylated PAPPA promoter and exhibiting the mitotic delay phenotype were not expressing PAPPA protein (FIGS. 7B and 7C).

Loss of PAPPA Expression is Linked to Tumour Progression

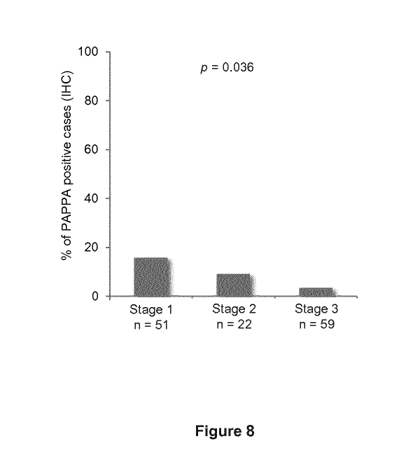

Immunostaining profiles showed PAPPA protein expression in 52% (13 out of 25 patients) of benign ovarian lesions, 31% (11 out of 36 patients) of borderline ovarian carcinomas and 11% (20 out of 184 patients) of invasive ovarian carcinomas (FIG. 7B-C). This indicates a significant trend showing a link between loss of PAPPA protein and tumour progression. PAPPA expression also inversely correlated with increasing tumour stage (FIG. 8). The inventors showed that 16% (8 out of 51 patients) of stage 1 ovarian carcinomas expressed PAPPA, compared to 9% (2 out of 22 patients) of stage 2 and 3% (2 out of 59 patients) of stage 3 carcinomas (FIG. 8). This trend was significant and indicates that PAPPA loss correlates with progression of ovarian cancer.

PAPPA Epigenetic Silencing is Linked to an Increase in the Proportion of Ovarian Cancer Cells Showing Mitotic Delay

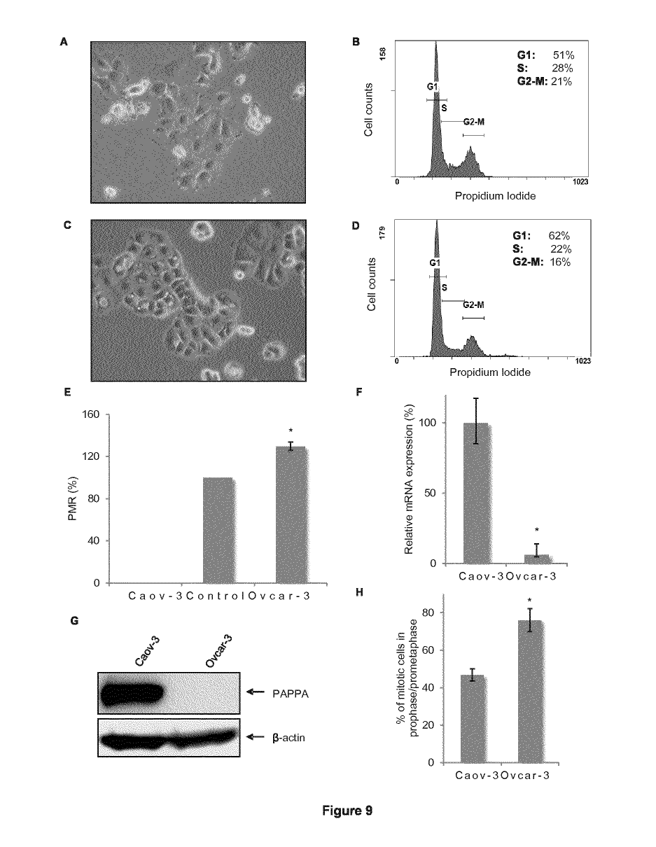

In order to corroborate the hypothesis that PAPPA loss is associated with mitotic delay in ovarian cancer an experimental in vitro model was required. Therefore it was investigated whether in ovarian cancer cell lines a link exists between PAPPA loss and the mitotic delay phenotype. Ovarian cancer cell lines Caov-3 and Ovcar-3 (FIG. 9A-D) were chosen for detailed study after determining PAPPA mRNA and protein levels (FIG. 9F-G). Mitotic phase distribution was determined by morphological analysis of H3S10ph immunostaining. The Caov-3 cell line has no detectable methylation of the PAPPA promoter and expresses PAPPA at mRNA and protein level (FIG. 9E-G). H3S10ph immunostaining demonstrated some mitotic delay (˜47% of the total mitotic cells in prophase/prometaphase) for this cell line (FIG. 9H). In keeping with the inventors' in vivo findings, a cell line (Ovcar-3) with hypermethylated PAPPA promoter did not express PAPPA protein (FIG. 9E-G) and exhibited a strong mitotic delay phenotype with ˜76% of mitotic cells in prophase/prometaphase (FIG. 9H).

PAPPA Loss Renders Ovarian Cancer Cells More Invasive

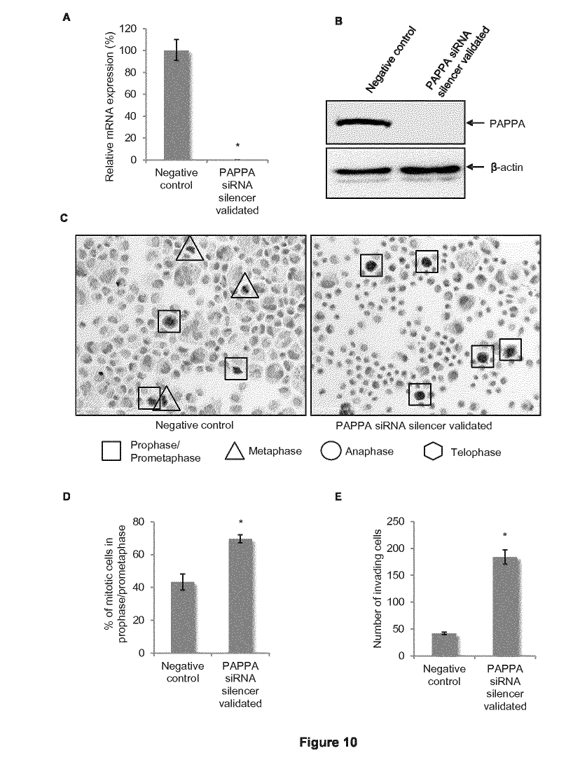

Next the inventors asked what biological advantage might be conferred to the neoplastic ovarian cell through perturbation of early mitotic progression. To address this question the inventors looked for any correlation between the mitotic delay phenotype and standard clinico-pathological features determined for each ovarian cancer specimen during routine clinical investigation (n=154 evaluable patients). This analysis did not reveal any linkage between mitotic delay and age of patient, tumour differentiation (grade), or morphological subtype (serous, endometrioid, mucinous, clear cell). The presence of the mitotic delay phenotype in nearly all invasive ovarian cancer specimens studied (96%, 148 out of 154 evaluable patients) raises the possibility that this mitotic defect might be linked to the acquisition of invasiveness. To test this hypothesis, the inventors induced the mitotic delay phenotype in Caov-3 cells by siRNA mediated PAPPA knockdown and measured the invasiveness of the manipulated cells in Matrigel-coated Boyden chamber assays. Relative to control siRNA, transfection of Caov-3 cells with PAPPA targeting siRNA resulted in significantly decreased PAPPA mRNA and protein expression (FIG. 10A-B). Consistent with the PAPPA knockdown, Caov-3 showed a significant increase in the proportion of mitotically delayed cells (FIG. 10C-D). Notably, experimental perturbation of mitotic progression in this cell model was associated with a significant increase in the number of cells invading through the matrix of a Boyden chamber (FIG. 10E). These results show that delayed progression through mitosis resulting from PAPPA downregulation increases the invasive capacity of ovarian cancer cells.

PAPPA Overexpression Reverses the Mitotic Delay Phenotype in Ovarian Cancer Cells

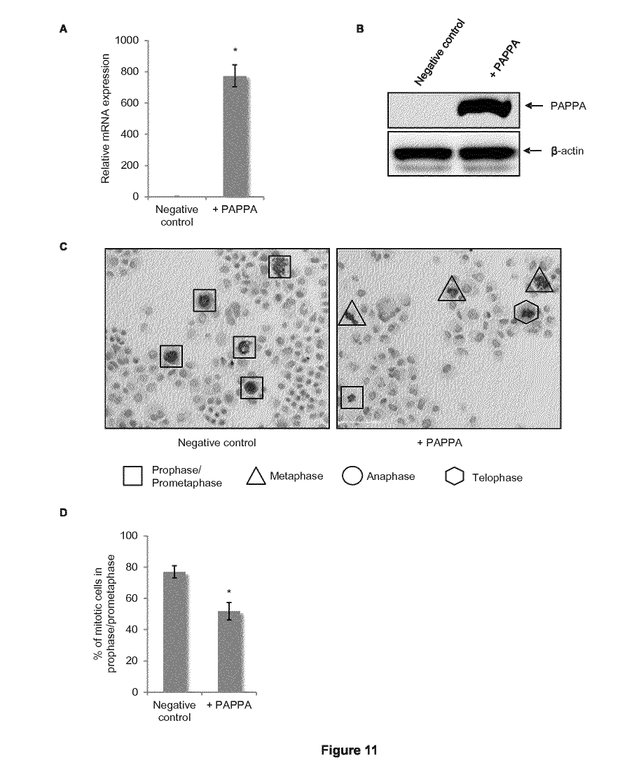

Since loss of PAPPA expression led to mitotic delay, it was hypothesised that PAPPA overexpression in a PAPPA negative cell line would reverse the mitotic delay phenotype. PAPPA was significantly overexpressed in Ovcar-3 cells compared to control transfections (FIG. 11A-B). Analysis of mitotic phase distribution in PAPPA overexpressing cells confirmed there was a decrease in the proportion of mitotic cells in prophase/prometaphase (FIG. 11C-D). In summary, the results in ovarian cancer cell lines demonstrate that PAPPA is required for progression through mitosis and that PAPPA downregulation through epigenetic silencing (Ovcar-3 cells) or experimentally by siRNA knockdown (Caov-3 cells) causes mitotic delay.

PAPPA Affects Mitotic Progression of Ovarian Cancer Cells by Regulating IGF-1 Signalling

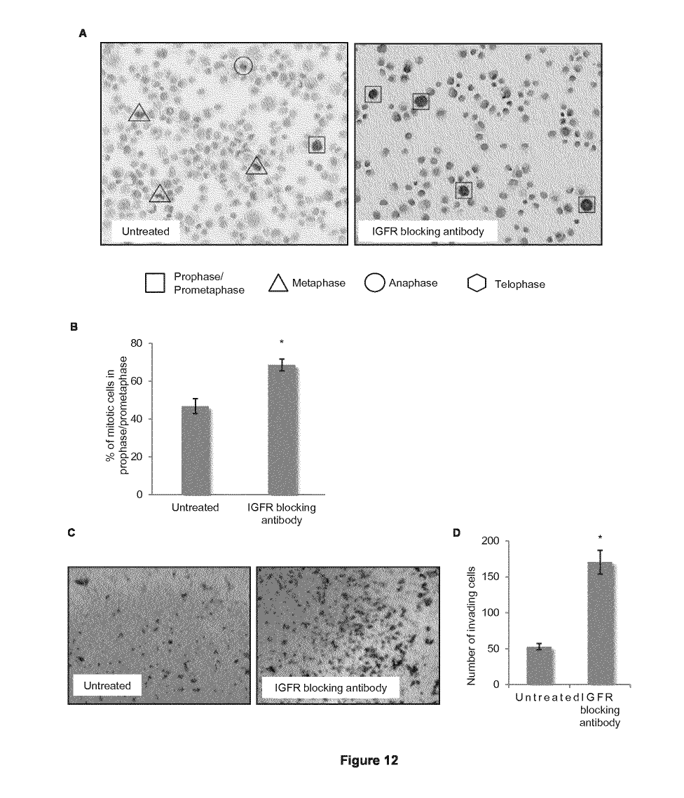

PAPPA-induced IGFBP-4 proteolysis increases the bioavailability of IGF-1 (H. B. Boldt, C. A. Conover, Growth Hormone & IGF Research 17 2007 10-18). Therefore it was further investigated whether experimental manipulation of the IGF-1 signalling pathway would affect mitotic progression and invasion. Initially the effect of inhibiting the IGF-1 receptor (IGF1R) using an IGFR blocking antibody in the Caov-3 cell line was investigated. Compared to untreated cells, Caov-3 cells treated with an IGFR blocking antibody demonstrated a significant increase in mitotic delay (FIG. 12A-B). Treatment of Caov-3 cells with IGFR blocking antibody also resulted in an increase in the number of invading cells as determined by Boyden chamber assay (FIG. 12C-D). These results suggest that blocking the IGF-1 receptor phenocopies the mitotic delay phenotype observed in Caov-3 cells after PAPPA knockdown.

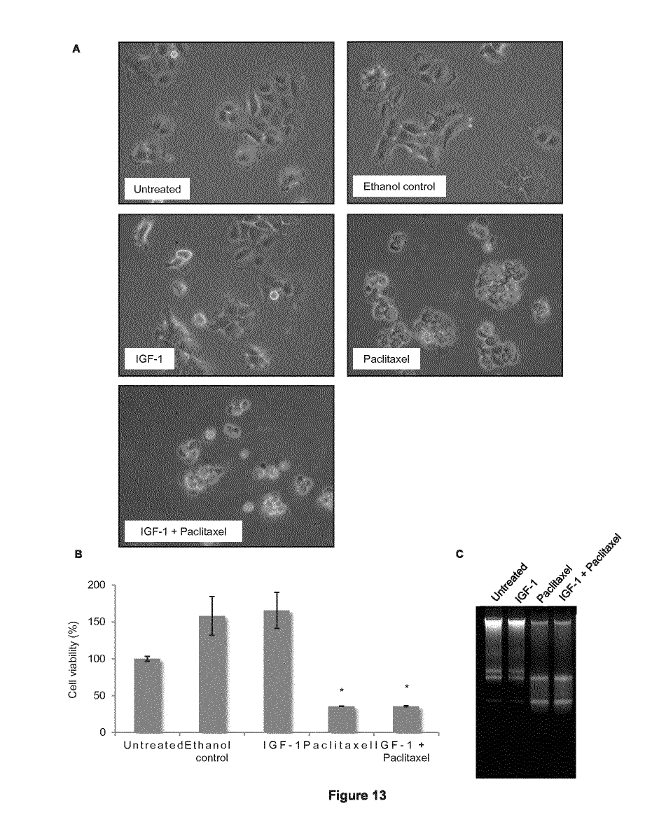

Paclitaxel is a chemotherapeutic drug given to treat ovarian, breast and non-small cell lung cancer. Paclitaxel binds to microtubules and prevents their breakdown. The movement of the replicated chromosomes during mitosis requires both polymerization of tubulin to form microtubules as well as the breakdown of those microtubules. In the presence of paclitaxel, chromosomes are unable to move to opposite sides of the dividing cell because microtubules are not broken down. Cell division is halted, and cell death is induced. Therefore it was hypothesised that ovarian cancer cells that were progressing normally through the cell cycle, such as Caov-3, would be particularly sensitive to paclitaxel treatment. The sensitivity of Caov-3 to paclitaxel was confirmed by cell morphology (FIG. 13A), decreased cell viability as measured by XTT assay (FIG. 13B) and induction of apoptosis as determined by DNA apoptosis kit (FIG. 13C). Exogenously added recombinant IGF-1 had no effect on cell viability in this line when added alone or in combination with paclitaxel.

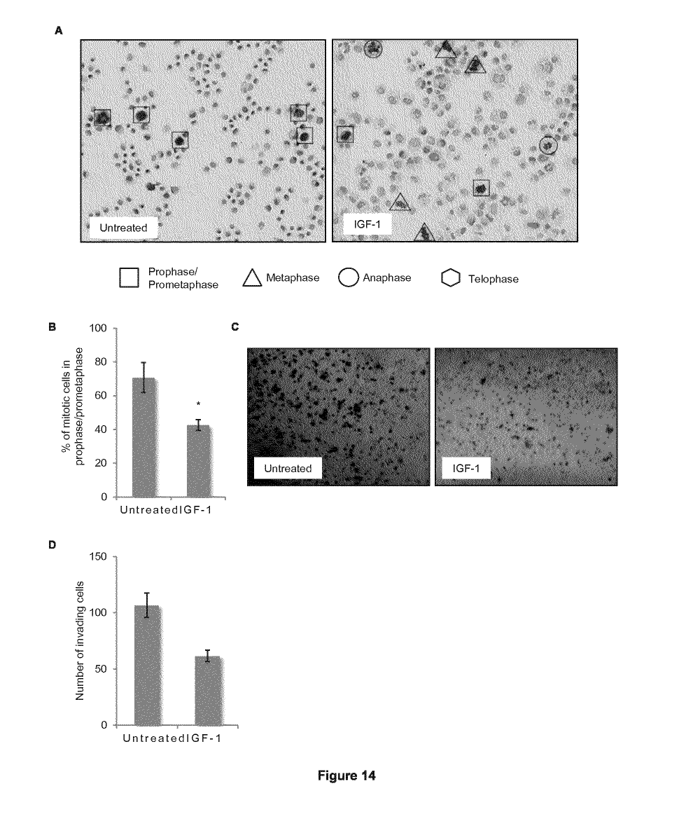

It was further hypothesised that pre-treatment of Ovcar-3 ovarian cancer cells, which closely resemble tumour cells in vivo (i.e. are characterised by PAPPA promoter methylation and show mitotic delay), with exogenously added IGF-1 should restore normal progression through mitosis in this cell line. Indeed, IGF-1 addition to Ovcar-3 cells led to a decrease in mitotic delay (FIG. 14A-B). In addition to restoring normal mitotic progression, exogenous also IGF-1 reduced the invasiveness of Ovcar-3 cells compared to untreated cells (FIG. 14C-D).

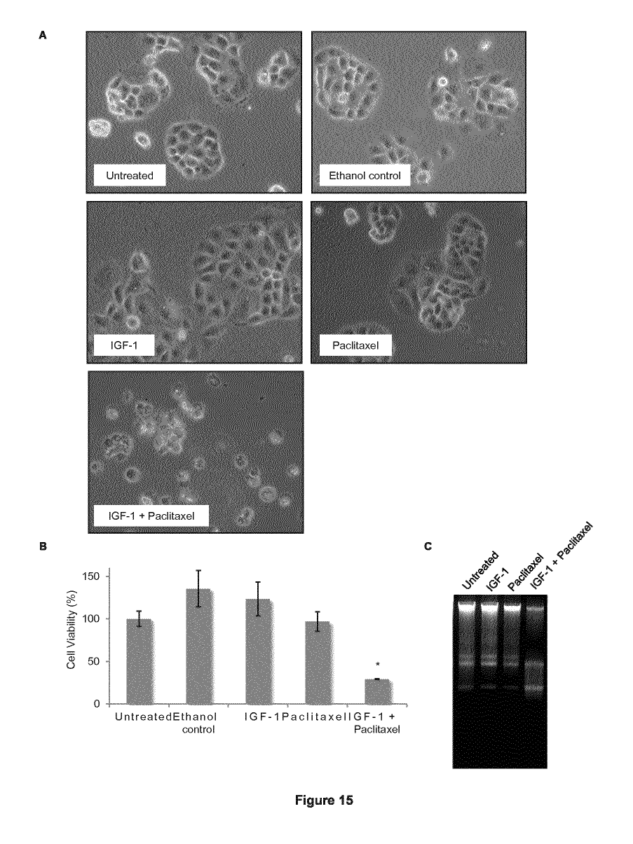

Finally, it was postulated that an ovarian cancer cell line which was mitotically delayed and resistant to paclitaxel treatment, would become sensitive to an anti-mitotic drug like paclitaxel after pretreatment with IGF-1 to restore normal mitotic progression. Supporting this notion it was found that Ovcar-3 cells were not responding to paclitaxel treatment at concentrations 5x greater than that which caused apoptosis in Caov-3 cells (FIG. 15A-C). However, when Ovcar-3 cells were treated with recombinant IGF-1 before the addition of paclitaxel, there was a significant decrease in cell viability as determined by XTT assay (FIG. 15B). This decrease in cell viability was linked to induction of apoptosis as determined with a DNA apoptosis kit (FIG. 15C). These results suggest that PAPPA affects mitotic progression by regulating IGF-1 signalling. The phenotypic read-out of PAPPA expression and progression through mitosis therefore provides a way to predict whether the cancer cell will be sensitive to therapy targeting the cell cycle. The data presented here suggest that therapies for PAPPA negative ovarian cancer could be fine-tuned for sequential treatment with a first agent (for example IGF-1) that removes the mitotic stalling and sensitises the tumour cells to a second drug (for example paclitaxel) that targets cells actively traversing through cell cycle (i.e. cells in G1 phase, S phase and G2/M phase of the mitotic cell division cycle).

1-68. (canceled)

69. A method for screening a subject for ovarian cancer, said method comprising:
detecting presence of a loss-of-function-related genetic alteration in the PAPPA gene or its regulatory or promoter sequences in a biological sample obtained from said subject; and
determining that the subject has ovarian cancer if presence of said loss-of-function-related genetic alteration in the PAPPA gene or its regulatory or promoter sequences is detected.

70. The method of claim 69, wherein said loss-of-function-related genetic alteration in the PAPPA gene or its regulatory or promoter sequences is one or more of a point mutation, deletion, insertion, translocation, chromosomal breakage, loss of heterozygosity or methylation.

71. The method of claim 70, wherein the presence of said loss-of-function-related genetic alteration in the PAPPA gene or its regulatory or promoter sequences is identified using a probe for the PAPPA gene or a specific mutation in the PAPPA gene.

72. The method of claim 70, wherein said loss-of-function-related genetic alteration in the PAPPA gene or its regulatory or promoter sequences causes a decrease in expression of the PAPPA gene, and wherein the presence of said loss-of-function-related genetic alteration in the PAPPA gene or its regulatory or promoter sequences is identified using a PAPPA-specific antibody or a probe for PAPPA mRNA.

73. The method of claim 69, wherein said subject is undergoing routine screening and is asymptomatic for ovarian cancer.

74. A method for aiding primary diagnosis of ovarian cancer in a subject suspected of having ovarian cancer, said method comprising the steps of:
detecting presence of a loss-of-function-related genetic alteration in the PAPPA gene or its regulatory or promoter sequences in a biological sample obtained from said subject; and
determining that the subject has ovarian cancer if presence of said loss-of-function-related genetic alteration in the PAPPA gene or its regulatory or promoter sequences is detected.

75. The method of claim 74, wherein said loss-of-function-related genetic alteration in the PAPPA gene or its regulatory or promoter sequences is one or more of a point mutation, deletion, insertion, translocation, chromosomal breakage, loss of heterozygosity or methylation.

76. The method of claim 75, wherein the presence of said loss-of-function-related genetic alteration in the PAPPA gene or its regulatory or promoter sequences is identified using a probe for the PAPPA gene or a specific mutation in the PAPPA gene.

77. The method of claim 75, wherein said loss-of-function-related genetic alteration in the PAPPA gene or its regulatory or promoter sequences causes a decrease in expression of the PAPPA gene, and wherein the presence of said loss-of-function-related genetic alteration in the PAPPA gene or its regulatory or promoter sequences is identified using a PAPPA-specific antibody or a probe for PAPPA mRNA.

78. The method of claim 74, wherein said biological sample is selected from the group consisting of ascites, blood, peritoneal fluid or washing, cystic fluid, smear and ovarian or fallopian tube tissue.

79. A method for determining that an atypical proliferative epithelial lesion or tumor is at risk of progressing to invasive ovarian cancer or that non-invasive ovarian cancer is at risk of recurrence, said method comprising the steps of:
detecting presence of a loss-of-function-related genetic alteration in the PAPPA gene or its regulatory or promoter sequences in a biological sample obtained from a subject who has an atypical proliferative epithelial lesion or tumor or non-invasive ovarian cancer; and
determining that the atypical proliferative epithelial lesion or tumor is at risk of progressing to invasive ovarian cancer or that the non-invasive ovarian cancer is at risk of recurrence if presence of said loss-of-function-related genetic alteration in the PAPPA gene or its regulatory or promoter sequences is detected.

80. The method of claim 79, wherein said loss-of-function-related genetic alteration in the PAPPA gene or its regulatory or promoter sequences is one or more of a point mutation, deletion, insertion, translocation, chromosomal breakage, loss of heterozygosity or methylation.

81. The method of claim 80, wherein the presence of said loss-of-function-related genetic alteration in the PAPPA gene or its regulatory or promoter sequences is identified using a probe for the PAPPA gene or a specific mutation in the PAPPA gene.

82. The method of claim 80, wherein said loss-of-function-related genetic alteration in the PAPPA gene or its regulatory or promoter sequences causes a decrease in expression of the PAPPA gene, and wherein the presence of said loss-of-function-related genetic alteration in the PAPPA gene or its regulatory or promoter sequences is identified using a PAPPA-specific antibody or a probe for PAPPA mRNA.

83. The method of claim 79, wherein said biological sample is selected from the group consisting of ascites, blood, peritoneal fluid or washing, cystic fluid, smear and ovarian or fallopian tube tissue.

84. The method of claim 83, wherein said biological sample is ovarian or fallopian tube tissue that exhibits proliferative pre-malignant epithelial tumor or lesions.

85. The method of claim 84, wherein the proliferative pre-malignant epithelial tumor or lesions comprise borderline tumors and/or intraepithelial neoplasms.


SEQUENCE LISTING (8 sequences)

SEQ ID NO: 1 - DNA, 22 bp, Artificial Sequence
  Description of Artificial Sequence Synthetic primer
cagaggagcccaagccaaagag

SEQ ID NO: 2 - DNA, 21 bp, Artificial Sequence
  Description of Artificial Sequence Synthetic primer
cacagccgtcccagtcacagt

SEQ ID NO: 3 - DNA, 21 bp, Artificial Sequence
  Description of Artificial Sequence Synthetic probe
gcgtgtttgtgcgagagttgt

SEQ ID NO: 4 - DNA, 21 bp, Artificial Sequence
  Description of Artificial Sequence Synthetic primer
cgccttccgaatatacccatt

SEQ ID NO: 5 - DNA, 24 bp, Artificial Sequence
  Description of Artificial Sequence Synthetic primer
tcgcccgaatatctctacgccgct

SEQ ID NO: 6 - DNA, 28 bp, Artificial Sequence
  Description of Artificial Sequence Synthetic probe
tctaacaattataaactccaaccaccaa

SEQ ID NO: 7 - DNA, 25 bp, Artificial Sequence
  Description of Artificial Sequence Synthetic primer
gggaagatgggatagaagggaatat

SEQ ID NO: 8 - DNA, 36 bp, Artificial Sequence
  Description of Artificial Sequence Synthetic primer
ccttcattctaacccaatacctatcccacctctaaa